(19) United States
(12) Patent Application Publication
(10) Pub. No.: US 2024/0345076 A1
(43) Pub. Date: Oct. 17, 2024

(54) Nanostructured Biomimetic ACE2 Sensors Based on a Superconductive Josephson Junction Toroidal Array Oscillating Effect for Speeding-up Screening of S1 SARS-CoV2 Inhibitors and Methods of Making the Sensor Thereto

(71) Applicant: Ellen T Chen, Rockville, MD (US)

(72) Inventor: Ellen T Chen, Rockville, MD (US)

(21) Appl. No.: 18/636,619

(22) Filed: Apr. 16, 2024

Related U.S. Application Data
(63) Continuation-in-part of application No. 17/364,348, filed on Jun. 30, 2021.

(51) Int. Cl. G01N 33/543; G01N 33/569

(52) U.S. Cl. CPC G01N 33/54373; G01N 33/56983; G01N 2333/948; G01N 2500/20

(57) ABSTRACT

A nanostructured biomimetic angiotensin-converting enzyme 2 (ACE2) function device was invented comprised of a superconductive Josephson toroidal junction array (JTJA). The device accurately detects a single particle Si SARS-CoV2 virus from 40 aM concentration up to 120 nM using multiple methods under antibodies-free and labeling-free conditions. The device is intended for speedy-screening candidate virus inhibitors through the JTJA with a S-I₁-S·I₂ (virus)·S (inhibitor) configuration to test whether or not a virus inhibitor could eliminate the virus's communication compared with a S1 inhibitor Remdesivir, and against a native ACE2 sensor. Results show the inhibitor ABS02 and Remdesivir effectively blocked virus communication through a flux quantum induced potential energy under an external magnetic field-free condition with the original memristive state transitioned to a superconductive state. The inhibitors restore a cell's reversible membrane potential with 100% efficacy in the safety zone compared to only 50% efficacy without the inhibitors.

### REFERENCE TO RELATED APPLICATIONS

This non provisional patent application U.S. Ser. No. 18/636,619 entitled of Nanostructured Biomimetic ACE2 Sensors Based on a Superconductive Josephson Junction Toroidal Array Oscillating Effect for Speeding-up Screening of S1 SARS-CoV 2 Virus Inhibitors and Methods of Making the Sensor Thereto is a Continuation in Part of U.S. non-provisional patent application Ser. No. 17/364,348 in the title of Nanostructured Model Devices of Making and Applications in Monitoring of Energy Landscapes of Toxic Protein Refolding Thereto that claims the benefit of U.S. Non Provisional patent application Ser. No. 17/364,348, filed on Jun. 30, 2021, and claims the benefit of U.S. Provisional Patent Application Ser. No. 63/471,566 filed in the title of Nanostructured Biomimetic ACE2 Memristive Electrochemical Sensors for Screening of a S1 SARS CoV-2 Inhibitor Candidate Compared with the Performance of Remdesivir filed on Jun. 7, 2023, and claims the benefit of a U.S. Provisional patent application No. 63/521,149, in the same title as U.S. 63/471,566, and filed on Jun. 15, 2023. The entire disclosure of the prior patent application Ser. No. 17/364,348 is hereby incorporated by reference, as is set forth herein in its entirety

### RELATED APPLICATIONS

The invention “Nanostructured Model Devices of Making and Applications in Monitoring of Energy Landscapes of Toxic Protein Refolding Thereto” is an electrochemical biosensor. It specifically refers to a device that functions as both a memristor and memcapacitor, serving as a dual function biosensor for detecting a biomarker directly linked to Alzheimer's disease and other neurodegenerative diseases. This invention is based on the principle of moonlighting proteins of HSP/MMP's capability to sense the energy change during a biomarker protein's refolding. The invention provides a unique nanostructured membrane fabrication method to build the device, and identify an antibiotic drug to fully block toxic protein Aβ refolding compared to the control in the cavity of HSP/MMP superlattices. It was not depending on the antibiotic drug's concentration, wherein to be able to maintain the Reversible Membrane Potential (RMP).

The instant invention “Nanostructured Biomimetic ACE2 Sensors Based on a Superconductive Josephson Junction Toroidal Array Oscillating Effect for Speeding-up Screening of S1 SARS-CoV 2 Virus Inhibitors and Methods of Making the Sensor Thereto” reported here falls within the field of quantum and memristive sensing of the toxic virus S1 SARS-CoV2. It specifically refers to a model sensor invented for the purpose of speeding up the screening of virus inhibitors. The principle behind this invention is that an originally mems-element sensor, that means the memristive, memcapasitive and meminductive, changes its state from a mems-element state to a superconductive quantum state as a Josephson toroidal junction array oscillator oscillating in the presence of a suitable S1 inhibitor at a higher scan rate ≥10 kHz (in a THz Josephson frequency range), because the S1 inhibitor induced a Josephson junction oscillation in a physiological media before and after S1 virus interacts with the sensor completely blocked the functional group of the biomimetic ACE2 membrane due to the continuing phase change of the inhibitor's sine waves. The inventor envisioned, that no virus, no cancer cells can survive at an environment of a voltage-controlled sine wave current takes phase change frequently at zero-bias, led a super-positioning effect. When a toxic virus interacts with the biomimetic ACE2 sensor in the presence of a suitable S1 inhibitor, such as our in-house developed ABS02 S1 inhibitor, regardless the virus is in a single particle concentration, or 120 nM concentration, the quantum oscillator due to the presence of the S1 inhibitor, could be fully blocking virus' communication compared with an established and the US FDA approved S1 SARS-CoV 2 inhibitor Remdesivir, as a reference S1 SARS-COV2 inhibitor material for comparison. Therefore, a suitable S1 virus inhibitor can be identified. By comparing with a native protein ACE2 sensor, as Sensor 2, results show ABS02 and Remdesivir inhibitors blocked S1 virus 100% and 90.9% for ABS02 and Remdesivir, respectively. Both inhibitors can restore the function of cell reversible membrane potential (RMP) by having 100% data (ratio of Action potential vs. Resting potential, Ap/Rp) located in the safety zone by blocking S1 over the range from 40 aM to 120 nM under antibody-free, tracer-free, and reagent-free conditions, compared 50% data outside of the safety zone for S1 virus alone without the inhibitor.

### BACKGROUND OF THE INVENTION

Protein moonlighting is a phenomenon that proteins perform two or more unrelated functions that are directly impacting human health [1-5]. MMPs and HSPs were well-known moonlighting proteins. Originally MMPs is known for their localization at the extracellular matrix (ECM), and have the role of degrading ECM proteins [1-5], but accumulated literature reported MMPs have been found in every cell compartment, such as in cytoplasm, in cell nuclei, and in mitochondria playing roles in apoptosis, tumor invasion, genetic instability, and innate immunity functionalities [1-7]. HSPs/MMPs working as a team to influence our immune system have been reported based on their moonlighting capabilities and unique behaviors [1-7]. This moonlighting catastrophic event may cause vulnerable to cancer patients, diabetes, coronary artery disease patients, and Alzheimer's patients when an unusual viral attacked, like SARS-CoV 2 viral in the pandemic, that a report had shown 50% upregulated genes among the top 10 infected human genes are belong to HSP family in the Covid 19 cases [8]. β-Amyloid and hyperglycemia can activate MMP-2 in the mitochondrial cell causing MMP-2 concentration increase, and it decreases the Heat Shock Protein (HSP) 60's concentration, which leads to disturbing the mitochondrial gap membrane potential, causes mitochondria cell dysfunction and released cytochrome c to apoptosis immune cells, hence, protein MMP/HSP network moonlighting contributes to many diseases [1-10].

Fluoroquinolones, levofloxacin, and moxifloxacin (MOX) prove to be clinically beneficial as adjunct treatment therapeutic agents for the management of severe Covid 19 patients worldwide according to reports in the literature [11-14]. There is very few, if any, to study the links between the Moon-lighting protein network of HSP/MMP with the proven effective antibiotics, such as moxifloxacin interacting with Aβ because of the high percentage of the mortality rate of Covid 19 is elderly who had significant underline diseases of Alzheimer's and dementia. The initial goal of this research project is to develop an HSP60/MMP-2 model device for evaluation of the MOX effectiveness to impair HSP60's function and lead to recover the reversible membrane potential in the presence of impact from Aβ compared with an HSP control device. Our prior research reported an innate HSP60/MMP-2 network protein device with cross-linked polymers forming superconductive and memristive nanostructured toroidal-tower array self-assembled membrane (SAM), was able to direct ultra-sensitively sensing multiple biomarkers, such as glucose, pyruvate, acetyl CoA, and choline, under antibody-free, label-free and tracer-free conditions [15]. The evidence implied that the HSP/MMP device mimicked the moon-lighting protein HSP/MMP network's characteristics. Under this discovery, we attempted to put this system under testing of its biocommunication with moxifloxacin with or without the impact of Aβ. Following Sections, we explain the methods used for evaluation of the protein refolding landscape energy changes with or without MOX in the presence of Aβ under antibody-free, labeling-free, reagent-free, and tracer-free conditions.

### Background of the Instant CIP Invention

Serological and immunological assay methods are widely used for the analysis of the presence of immunoglobulin (IgM) or IgG antibodies in blood serum or testing in posterior oropharyngeal saliva for public screening testing during the Covid 19 pandemic [1-3]. Spike (S) protein has been a well-known target antigen of the SARS-CoV-2 virus, that first attacks human angiotensin-converting enzyme 2 (ACE2) receptor-binding domain (RBD), then mediating entry into human cells [4-7]. Fast and precise detection of the presence of SARS-CoV-2 virus using the gold standard of reverse transcription-polymerase chain reaction (RT-PCR) is very challenging in order to meet the urgent needs, due to the performance limitations of the instrumentation, expensive reagents, long time waiting for results, and protein interference [1-4]. The American Society for Microbiology COVID-19 International Summit had suggested further improving the testing of SARS-CoV-2 viral antigens considered to deserve further research for Point-of-Care (POC) facilities to test asymptomatic patients [8]. Another call from the article revealed the fact that there is a worldwide shortage of reagents to perform the detection of SARS-CoV-2. Many clinical diagnostic laboratories rely on commercial platforms that provide integrated end-to-end solutions. While this provides established robust pipelines, there is a clear bottleneck in the supply of reagents given the current situation of extraordinarily high demand [9]. In an attempt to respond to urgent calls and to fulfill the unmet needs, the aims of this invention are to develop a hand-hold electrochemical S1 antigen sensor for fast, accurate sensing and monitoring of single-particle SARS-CoV-2 virus in human biological specimens under reagent-free, label-free and antibody-free conditions.

The speedy screening for chosen pharmaceutical candidates, especially for S1 SARS-COV2 inhibitors, has been in high demand, and the processing faces challenges, such as virus mutations created challenges for vaccine and drug development, and identify a suitable pharma candidate is very time consuming because researchers have to screening thousands of candidates, furthermore is limited knowledge about the pathophysiology of the virus, inducing humoral or cellular immunity, immune enhancement with animal coronavirus vaccines, and lack of higher thoughput animal disease models for the selection of candidate vaccines [10]. Our group developed S1 SARS-CoV2 antigen sensors with embedded native ACE2 receptor protein at concentrations over 4.5, 57.5 to 230 nM, respectively, and cross-linked with multiple copolymers formed self-assembled membranes under antibody-free conditions for sensors 1, 2, and 3, respectively for fast testing and monitoring of a single S1 particle SARS-CoV-2 virus in 120 s, which is suitable for testing asymptomatic patients with higher than 92±9% to 96±4% accuracy using spiked 40 aM fasting human saliva specimens against the results from the calibration curves by the DSCPO and an open circuit potential (OPO) method, respectively. Low ACE2 concentration embedded in sensor 1 has the results of reversed potential membrane (RPM) indicator of the ratio of action potential/resting potential (Ap/Rp) located in the unsafe zone, while sensor 2 and 3 kept in the normal zone. Accuracy results are 99±2% using spiked S1 60 nM in NIST SRM965 human serum compared with that of the data from calibration. The imprecision results are 1.96% over the linear range from 0.1 nM to 100 nM (n=12, p<0.0001); the imprecision result of the single virus particle has an RSD 0.1% related to the mean over the concentration range from 5 aM to 100 pM (n=18, p<0.0001) using the OPO method under label-free, and antibody-free conditions [11]. Our invention will revolutionize the way electrochemical sensing systems work, and it will have a significant impact on detecting the S1 SARS-CoV 2 virus, which is the need of the hour.

### BRIEF STATEMENT OF THE PRIOR ART

The prior art revealed using an electromagnetic wave at high frequency to kill S1 SARS COV 2 virus by applying 2 min of 2.45 GHz, 700 W electromagnetic radiation could denature SARSCoV-2 spike protein, which is critical for the entry of SARS-CoV-2 into host cells, through the formation of hot spots and the interaction of the oscillating electric fled with different parts of protein charges via a pure electromagnetic effect [12]. The drawback is the high watt and long-time radiation may cause safety issues if applied directly to humans, we understood their research goal is just to denature the virus in vitro, not to apply the RF wave directly on humans. Inspired by their approach, we propose to use a single flux quantum forming by a nanostructured biomimetic ACE2 memristive sensor when the S1 SARS-COV2 single particle virus interacts with the sensor, will be blocked its toxicity by an S1 inhibitor when the inhibitor presences, yet utilizes the virus as a Josephson barrier, hence promotes Cooper-pair delocalized tunneling inducing Josephson inductance of high frequency oscillating, and the non-magnetic power of the single flux quantum in the sensor not only deactivate the virus, but also gaining potential energy to the system under field-free, external magnetic field-free conditions without harm to the sensor.

### OBJECT AND SUMMARY OF THE INVENTION

It is an object of the invention to provide a new type of model device that is able to conduct real-time evaluation of the landscape energy change during the processing of protein folding with or without antibiotic under an influence of the biomarker beta-amyloid (Aβ) at an open circuit potential state under antibody-free, labeling-free, reagent-free, and tracer-free conditions.

It is an object of the invention to provide a new type of model device that is able to reveal the i-V curves of the direct electron transfer peak of an analyte at oxidation or reduction states at different scan rates of the analyte to a wide range concentration.

It is an object of the invention to discover a new type of fabrication technology with optimum compositions of polymers cross-linked with innate proteins, such as Heat Shock Protein (HSP) 60, forming a self-assembled membrane on the surface of gold sensor chip, that enables the device to sense protein refolding energy landscape change of a biomarker protein, such as beta-Amyloid (A3) in the presence of an antibiotic drug, such as moxifloxacin (MOX).

It is an object of the invention to discover a new type of fabrication technology with optimum compositions of conductive organic polymers having multiple functioning groups cross-linked with multiple innate proteins, such as Heat Shock Protein (HSP) 60 and Matrix Metalloproteinase (MMP)-2 forming multiple-layer self-assembled membranes on the surface of gold sensor chip to mimic a “Moonlighting-protein Network” in order to search for a better inhibitor to impair A3 refolding in the HSP cavity for the purpose of reduce elderly patients' vulnerability to the virus attack.

It is an object of the invention to discover a new method for monitoring of the Reversible Membrane Potential (RMP) after using the antibiotic drug MOX.

### Summary of the Instant CIP Invention

It is an object of the present invention to develop a highly efficient electrochemical sensing system that emulates the function of ACE2, called biomimetic ACE2 Sensor 1, that can detect S1 SARS-CoV2 virus over a wide range from a single particle to 120 nM concentration by the virus's electrochemical communication with Sensor 1 with or without the presence of a S1 SARS-CoV2 inhibitor candidate, and compare the inhibitor's effect to block the virus from communication with the sensor. The results will be compared with Remdesivir, an established S1 SARS-CoV-2 inhibitor approved by the FDA, to fast screen the suitable inhibitor. Our goal is to create a system that integrates the biomimetic ACE2 sensor, S1 SARS-COV2 virus, and S1 virus inhibitors as one system. This system will facilitate long-range tunneling of Cooper-pairs across a barrier of zinc ions, serving as the primary insulator, and across through the virus, acting as a secondary insulator, until the Cooper-pair synchronizes with the S1 virus inhibitor molecule and the sensor at a high frequency. This will form a Josephson toroidal junction array device with nanobiomimetic ACE2 function and anti-virus capabilities to identify the suitable virus inhibitor when a virus enters the system. If a wrong type of virus inhibitor presences in the system, there would be no superconductive oscillation at zero-bias. The result will be a superconductive quantum oscillation that suppresses the original sensor's capacitive energy by orders of magnitudes, and switches to a superconductive potential energy at zero-bias through the supercurrent oscillating due to frequently phase change. Through this process, the virus becomes a harmless component of the system. If this model succeeds, that would be opened a road to face the challenge of frequent virus variability, even extend to identify cancer inhibitors.

It is an object of the present invention to compare the results obtained from Sensor 1 with a native ACE2 sensor, Sensor 2, which has established a performance standard traceable to using NST standard human serum spiked with the virus for calibration curve development for accurate detection virus.

It is an object of the present invention to use multiple validating protocols, such as the cyclic voltammetry method (CV), Open Circuit Potential (OPO) method, and double-step chronopotentiometry (DSCPO) method to identify the S1 inhibitor for a fair comparison.

It is a further object of the present invention to judge the inhibitor's impact on a biological cell's reversible membrane potential (RPM) with spiked S1 antigen compared to the cell without using a virus inhibitor, using the clinical safety zone of the AP/RP ratio method.

It is a further object of the present invention to evaluate the S1 inhibitors under labeling-free, antibody-free, and tracer-free conditions with the change of the S1 SARS-CoV 2 concentration levels over aM to 120 nM concentrations.

It is a further object of the present invention to make the biomimetic ACE2 sensor more sensitive detection of the S1 SARS-Cov2 virus compared with a native ACE2 electrochemical memristive sensor.

It is a further object of the present invention to in-house develop an inhibitor for the S1 SARS-CoV2 virus and be able to block S1 virus communication with the biomimetic ACE2 sensor reaching nearly 100% efficiency compared with a native ACE2 sensor, herein the first invented quantum sensor utilized a toxic S1 SARS-COV2 virus protein as the secondary Josephson toroidal junction insulator that promotes a single flux quanta formation, which is perpendicular to the circular supercurrent flow surface through the S-I-S·S1 virus·S1 inhibitor configuration, wherein the S1 SARS-COV2 lost toxicity and identity in the complex, and the combination of the superconductive Josephson junction toroidal array oscillating effect and the Josephson effect led to the original memristive state switched to a superconductive quantum state is envisioned.

### BRIEF DESCRIPTION OF THE FIGURES

FIG. 1A depicts Sensor 1 of the HSP60 SAM's 3D image in an area of 1.0×1.0 μm² with a ±Z-value 259.3 nm. FIG. 1B depicts the 3D AFM image. FIG. 1C depicts the HSP60 cluster on the right-side corner, and the subunit cluster on the top left-side corner. FIG. 1D depicts the top view of the HSP60 nanopore rings and the side view of the HSP60 structure. FIG. 1E shows another cluster of HSP60 in a 1.4×1.4 μm² area, it emitted golden light beams from the HSP clusters. FIG. 1F shows the image of nanotube density differently oriented on the top-hill area and on the low-lay canyon area of the 2D AFM image.

FIG. 2A depicts the 3D AFM image of the MMP-2 toroidal array superlattice membrane as the first layer in Sensor 2. FIG. 2B depicts the 2D AFM image in a larger area. FIG. 2C depicts the second layer of tower structure HSP60 AFM image multiple-enzyme network membrane which is on top of the first layer of MMP-2. FIG. 2D depicts the toroidal “diamond ring” structure in detail. FIG. 2E depicts another AFM image from a different area for the tower glow with Cooper-pair moving with the Friedel-oscillation compared with the background superlattice of toroidal rings. FIG. 2F depicts the 2D AFM image of a HSP60 subunit as a donut at the low z-value 70 nm. FIG. 2G depicts the 3D AFM image of the tall nanometer size “Tesla Tower” with height between 500-700 nm.

FIG. 3A shows an art model for Device 2 of the superconductive/energy sensing device of HSP60/MMP-2 moonlighting-network in a side view of the Josephson Junction. “60” is the electrode; “61” is the amplified wave after the Cooper pair went through the multiple superconductor-Insulator (the mobile zinc ions from MMP-2)-superconductor (SIS) layers at a higher frequency. “62” refers to the Cooper pair; “63” refers to the circular current flow in a positive direction with the zinc atoms as the brown balls; “64” refers to the HSP6's double-truncated donut cavity array matrix alignment with each other produced the eternal superconducting current in the blue circle having induced a φ₀, single flux quantum by means of zinc ions bridged from the MMP-2, that a non-ferromagnetic field is produced; “65” is the nano-island membrane on the gold electrode; “66” is the wave of cooper pair electrons after passing through the nano-island membrane; Notice there is an air barrier between the membrane and the array of HSP60 matrix. “67” refers to the PEG . . . PVP's N-terminal chain. FIG. 30B depicts the detailed art model of the HSP60/MMP-2 with “1” refers to the gold electrode, “2” refers to the MMP-2 toroidal structure comprising of two small toroidal circuits in the center with zinc ions serve as the JJ's “insulator”, “3” reefs to the two pieces of HSP60's truncated double-donuts, “4” refers to the mobile zinc ions from MMP-2 up to the HSP cavity, promoting the long range superconducting current, and “5” refers to the Cooper pair electrons.

FIG. 4A depicts Sensor 1's i-V curves of controls over different scan rates. At 20 Hz, compared curves of MOX affecting Aβ. FIG. 4B depicts the contour map between the current of MEM_{red} and current of MEMₒₓ peak intensity vs. time of 10 consecutive scan at 1 kHz in the buffer with the sensor 1. It showed the highest correlation between the variables is at the higher scan cycles.

FIG. 5 depicts MOX affecting i-V curves of Aβ at 20 Hz (Panel A) and 20 kHz (Panel B) compared with controls.

FIG. 6 depicts Sensor 1's 250 ng/mL Aβ effects on the open circuit potential vs. 2-, 6-, and 12-minutes monitoring of the energy change compared with controls.

FIG. 7 depicts various concentrations of HOX impaired 250 ng/mL A3 folding on the open circuit potential vs. 2 and 6 minutes compared with controls, respectively.

FIG. 8 depicts Sensor 2 (HSP60/MMIP2)'s open circuit potential curves at 2 minutes monitoring of the energy change over 500 ng/mL to 80 μg/mL MOX in the presence of 250 ng/mL A3 compared with controls.

FIG. 9A depicts the potential vs. time curves at ±10 nA at MOX concentration from 10 to 80 μg/mL with or w/o Aβ at 250 ng/mL for Sensor 1 (sample run triplicates).

FIG. 9B depicts Sensor 2's profiles with MOX from 0.5 to 80 μg/mL (5 levels).

FIG. 10 depicts the comparison between Sensor 1 and Sensor 2 for the plots of AP/RP ratio vs. MOX concentration in the presence of 250 ng/mL Aβ over MOX from 0 to 80 μg/mL.

FIG. 11 is the content of Table 1, it compares the performances of two sensors by linear regression method of |Ap/Rp| vs. MOX concentration with 250 ng/mL Aβ.

FIG. 12A depicts the AFM image of the top layer superconductive superlattice membrane made with nanostructured organometallic 3D-caged toroidal structure comprising of bM-β-DMCD/TCD/PEG/PVP/Zinc Chloride with Friedel-oscillation observed in Sensor 1. FIG. 12B depicts Sensor 1's bottom layer AFM image of an organic conductive polymer nano-island membrane for sensor 1 comprising of TCD/PEG/PVP/Co-cyclodextrin (Co-CD).

FIG. 13 depicts the sensor 2's AFM image embedded with the native ACE2 protein 230 nM cross linked with multiple polymers.

FIG. 14A depicts the circuitry symbol of the Flexible Toroidal Josephson Junction Superconductive Quantum Interference Device (FTJJSQUID) comprising of at least 4 or more junctions and a self-powered switchable reversible electron-relay in the center which is the fundamental function of the biomimetic self-assembled membrane (SAM) that the MEMS-element construction relies on. FIG. 14B depicts the mM-β-DMCD, and bM-β-DMCD's cyclic supramolecule chemical structure.

FIG. 15A depicts the molecular structure of Remdesivir, which is a SARS-CoV-2 Nucleotide Analog RNA polymerase inhibitor. FIG. 15B depicts the 3D-cage structured S1 SARS-CoV2 inhibitor ABS02 comprised of cross-linked dM-β-DMCD, PEG, PVP, TCD, Zinc Chloride, cysteine, and collagen-1. FIG. 15C depicts a scheme of Sensor 1 detects S1 SARS-CoV2 virus with or without ABS02 S1 inhibitor in three steps: Step 1 depicts Senso1'a nanostructured membrane forms the Direct Electron Relay (DET) with a hemming bird-like hysteresis current loop when a voltage is applied and scanned. Step 2 depicts the membrane forms a chelating complex when the S1 virus particle. Step 3 depicts S1 virus selectively interacts with the inhibiter forms a long range DET relay helped Cooper-pair crosses the primary Josephson junction barrater of zinc ions, and the secondary barrier of the virus and induced a high frequency oscillation. The relay turns on the superconductive state after S1 virus strongly chelated to ABS02.

FIG. 16A depicts the i-V curves at different scan frequencies over 60 Hz to 10 kHz in the PBS control solution for the mems-element Sensor 1. FIG. 16B depicts the i-V curves of S1 SARS-COV 2 inhibitor ABS02 with concentration 0.8 nM with sensor1 without S1 SARS-CoV2 over scan rate 60-25 kHz. FIG. 16C depicts the i-V curves of the S1 SARS-COV 2 inhibitor Remdesivir with concentration 0.8 nM with sensor1 without S1 SARS-CoV2 presences over scan rate 60-25 kHz.

FIG. 17A depicts the i-V curves at different S1 SARS-CoV2 concentrations between 40 aM-120 nM at the scan rate 10 kHz using Sensor 1 of the biomimetic ACE2 sensor. FIG. 17B depicts the calibration curve of the S1 SARS-CoV2.

FIG. 18A depicts the i-V curves of S1 CoV2 virus over concentration range 40 aM-120 nM in the presence of the inhibitor ABS02 with a concentration 0.8 nM at scan rate 10 kHz using Sensor 1. FIG. 18B depicts the i-V curves of S1 CoV2 virus over concentration range 40 aM-120 nM in the presence of the inhibitor Remdesivir with a concentration 0.8 nM at scan rate 10 kHz. FIG. 18C depicts the semi log plots of superconducting current at V=0 vs. S1 SARS-CoV2 concentrations between 40 aM-120 nM in the presence of 0.8 nM ABS02, or 0.8 nM Remdesivir, respectively compared with the control calibration curve shown in FIG. 18B. ▪ refers to the backward scan in the presence of 0.8 nM ABS02, ⊕ refers to the forward scan in the presence of 0.8 nM ABS02; ▾ refers to the backward scan in the presence of 0.8 nM Remdesivir, and ▴ refers to the forward scan in the presence of 0.8 nM Remdesivir.

FIG. 19A depicts the i-V curves at different scan frequencies over 1 Hz to 10 kHz in the PBS control solution for the native ACE2 mems-element Sensor 2 comprising of protein ACE2/TCD/PEG/PVP SAM membrane on a 50 nm thickness gold chip as anode electrode, with the gold cathode electrode, and a reference electrode is Ag/AgCl. FIG. 19B depicts the i-V curves of different S1 SARS-CoV2 concentrations between 5 aM-120 nM at the scan rate 10 kHz using Sensor 2 of the protein ACE2 sensor without an inhibitor. FIG. 19C depicts the i-V curves of S1 SARS-CoV2 concentrations between 40 aM-120 nM at the scan rate 10 kHz using Sensor 2 of the protein ACE2 sensor in the presence of a 0.8 nM ABS02 of the S1 SARS-CoV2 inhibitor. FIG. 19D depicts the i-V curves of S1 SARS-CoV2 concentrations between 40 aM-120 nM at the scan rate 10 kHz using Sensor 2 of the protein ACE2 sensor in the presence of a 0.8 nM Remdesivir of the S1 SARS-CoV2 inhibitor.

FIG. 19E depicts the calibration curves of DETₒₓ current and DET_{red} current of the S1 SARS-CoV2 virus using the protein ACE2 Sensor 2 of the CV method over 40 aM-120 nM, respectively at 10 kHz scan rate. FIG. 19F depicts the calibration curves impacted of the DET_{red} current of the S1 SARS-CoV2 virus using the protein ACE2 Sensor 2 of the CV method over 40 aM-120 nM after 0.8 nM ABS02 inhibitor ( [symbol] ), and 0.8 nM Remdesivir (⊕) applied at 10 kHz scan rate, respectively. The calibration curve without S1 inhibitor using symbols [symbol] .

FIG. 20A depicts the plots of the signal of open circuit potential curves (the OPO method) after subtracted the background signal vs. time (s) for 120 s for S1 SARS-COV2 concentration over 40 aM-120 nM over 7 concentration levels against the PBS controls, with each sample run triplicates using the biomimetic ACE2 Sensor 1 before applying an inhibitor. FIG. 20B depicts the plots of the signal of open circuit potential after subtracted the background signal vs. time (s) for 120 s for S1 SARS-COV2 concentration over 40 aM-120 nM over 7 concentration levels against the PBS controls, with each sample run triplicates using the biomimetic ACE2 Sensor 1 after applying a 0.8 nM inhibitor Remdesivir samples. FIG. 20C depicts the plots of the signal of open circuit potential curves after subtracted the background signal vs. time (s) for S1 SARS-COV2 concentration over 400 aM-120 nM over 5 concentration levels against the PBS controls and the 0.8 nM ABS02 controls, respectively. Each sample run triplicates using the biomimetic ACE2 Sensor 1 after applying a 0.8 nM inhibitor ABS02 samples. FIG. 20D depicts the semi log calibration curves of the OPO potential of the S1 SARS-CoV2 virus using the biomimetic Sensor 1 of the OPO method over 40 aM-120 nM after 0.8 nM ABS02 inhibitor ( [symbol] ), and 0.8 nM Remdesivir (⊕) applied, respectively. The calibration curve without S1 inhibitor using symbols [symbol] .

FIG. 21A depicts the plots of the signal of open circuit potential curves (the OPO method) vs. time (s) for S1 SARS-COV2 concentration over 5 aM-100 pM over 6 concentration levels against the PBS controls, with each sample run triplicates using the protein ACE2 Sensor 2 before applying an inhibitor. FIG. 21B depicts the plots of the signals of open circuit potential curves vs. time (s) for S1 SARS-COV2 concentration over 0.1 nM-120 nM over 4 concentration levels against the PBS controls, with each sample run triplicates using the protein ACE2 Sensor 2 before applying an inhibitor. FIG. 21C depicts the semi log calibration curves of the OPO potential of the S1 SARS-CoV2 virus antigen using the protein ACE2 Sensor 2 of the OPO method over S1 virus 5 aM-100 pM before apply an inhibitor compared with that of the buffer controls each sample run triplicates. FIG. 21D depicts the calibration curves of the OPO potential of the S1 SARS-CoV2 virus antigen using the protein ACE2 Sensor 2 of the OPO method over S1 virus concentrations between 0.1 nM-120 nM before apply an inhibitor. Each sample run triplicates. FIG. 21E depicts the plots curves of the signal of open circuit potential vs. time (s) for S1 SARS-COV2 concentration over 40 aM-120 nM over 6 concentration levels against the PBS controls, with each sample run triplicates using the protein ACE2 Sensor 2 after applying a 0.8 nM inhibitor Remdesivir samples. FIG. 21F depicts the plots curves of the signal of open circuit potential vs. time (s) for S1 SARS-COV2 concentration over 400 aM-120 nM over 5 concentration levels against the PBS controls and the 0.8 nM ABS02 controls, respectively. Each sample run triplicates using the protein ACE2 Sensor 2 after applying a 0.8 nM inhibitor ABS02 samples. FIG. 21G depicts the semi log calibration curves of the OPO potential of the S1 SARS-CoV2 virus using the protein ACE2 Sensor 2 of the OPO method over 40 aM-120 nM after 0.8 nM ABS02 inhibitor ( [symbol] ), and 0.8 nM Remdesivir (⊕) applied, respectively. The calibration curve without S1 inhibitor for the experimental date using symbols [symbol] . The polynomial fitting for the calibration curve without using an inhibitor is the symbol of ▪. FIG. 21H further depicts the difference of the potential vs. time curves between the impact of 0.8 nM ABS02 and 0.8 nM Remdesivir for S1 SARS-CoV2 over the same range of concentration shown in FIG. 21G, respectively using the OPO method.

FIG. 22A depicts the S1 SARS-CoV2 standards cell voltage vs. time using the biomimetic ACE2 sensor 1 by the voltage method (the double-step chronopotentiometry) (DSCPO) without an inhibitor over S1 concentrations 40 aM-120 nM with 5 levels compared with buffer controls. Each sample run triplicates. The insert curves refer to that of the low S1 concentrations curves. FIG. 22B depicts the voltage vs. time curves in the presence of 0.8 nM Remdesivir over the same S1 concentration range between 40 aM-120 nM compared with controls. Each sample rum triplicates. FIG. 22C depicts the voltage vs. time curves in the presence of 0.8 nM ABS02 over the same S1 concentration range between 40 aM-120 nM compared with controls. Each sample rum triplicates.

FIG. 22D depicts the results for the restoration of the RMP used the biomimetic ACE2 sensor 1 by the voltage method, 0.8 nM ABS02 and 0.8 nM Remdesivir both (n=15 for each method) have ratio values 100% located in the safety zoon over S1 40 aM to 120 nM, but S1 SARS-CoV2 (n=18) alone has more than 50% data located outside of the safety zoon.

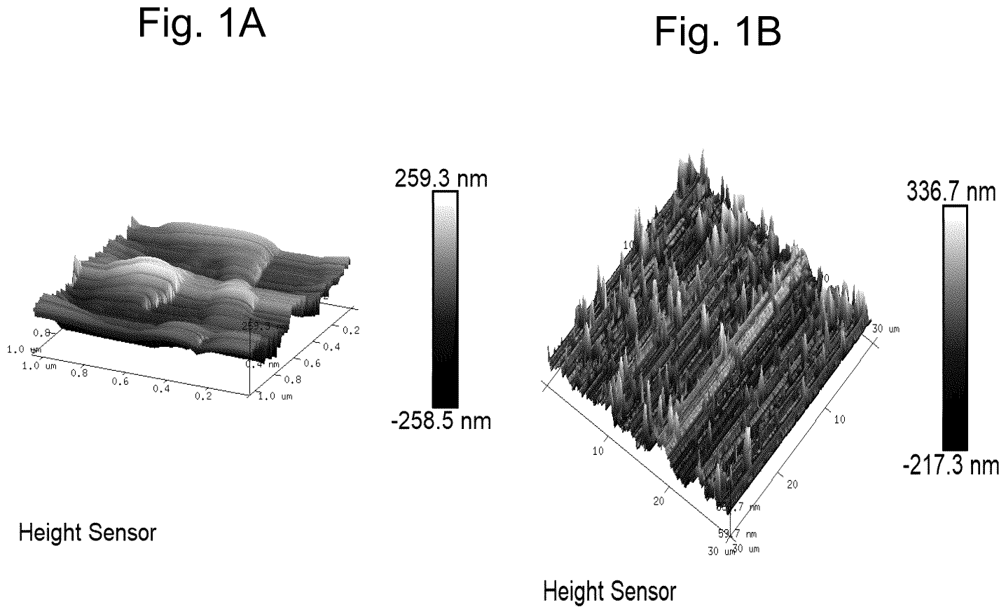

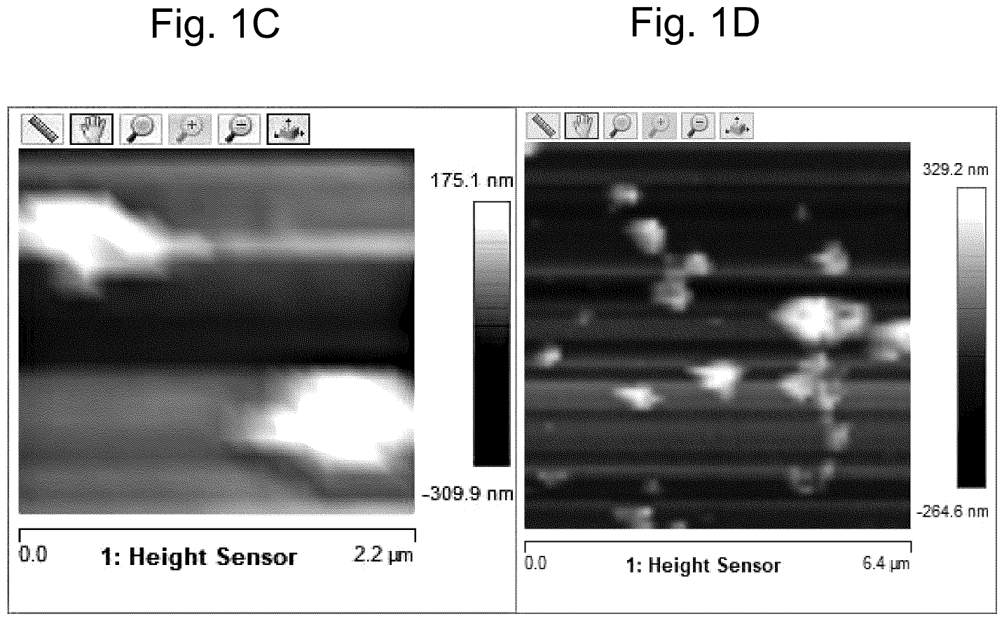

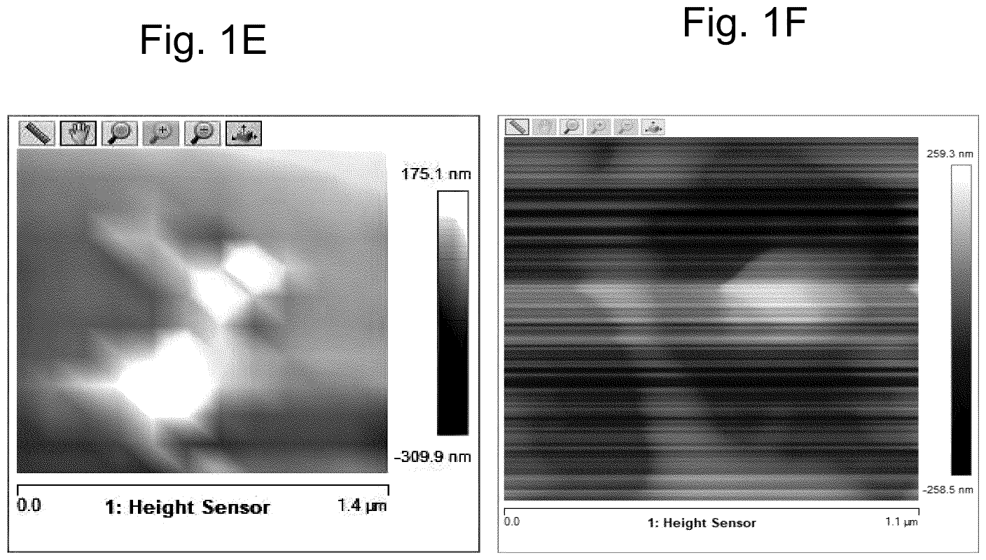

### DETAILED DESCRIPTION OF THE INVENTION

### Example 1—Fabrication of the Superconductive/Energy Sensing Device Having Superlattice Toroidal Structures

Sensor 1's membrane was fabricated by deposition of a mixture solution comprised of HSP60, triacetyl-β-cyclodextrin (TCD), polyethylene glycol diglycidyl ether (PEG), and poly (4-vinyl pyridine) (PVP) with appropriate propositions on the surface of gold chips at 37° C. for 72 hours. The procedures used for fabrication of the innate HSP60/MMP-2 self-assembled membrane was followed by published literature [15].

The device 2 for the innate HSP60/MMP-2 device was prepared with two steps: the first step was to form an MMP-2 polymer layer by a self-assembling method with compositions of the innate MMP-2, triacetyl-β-cyclodextrin (TCD), polyethylene glycol diglycidyl ether (PEG) and poly (4-vinylpyridine) (PVP) with appropriate propositions deposited on the surface of a 50 nm thickness gold chip having 16 channel plate, while each channel has three pure gold electrodes flatly layout sitting on a flexible non-conductive plastic plate with the plastic plat thickness less than 500 μm. The center working electrode was used for the polymer mixture to be deposited onto it at 37° C. for 96 hours after that followed the wash and dry procedures [15]. The auxiliary electrode has a circuit length 2.5-times longer than that of the working electrode's circuit length. The reference electrode is gold. The second layer was fabricated as same procedures for sensor 1. The MMP-2 was purchased from Ana Spec (Freemont, CA).

### Example 2—Characterization of the SAM Membranes

The morphology of the AU/SAM was characterized using an Atomic Force Microscope (AFM) (model Dimension Edge AFM, Bruker, MA). Data collected in Tapping Mode using silicon probes with 5-10 nm tip radius and ˜300 kHz resonance frequency (Probe mode TESPA-V2, Bruker, MA). FIGS. 28A and 28B refer to 3D AFM images of the multiple-layered SAMs of Sensor 1. FIG. 28B has many vertically oriented nano-pillars on top of horizontally oriented well-ordered high and low-lay densified nanotubes. FIG. 28C reveals a cluster of 37 two-heptametrical rings made up tetradecamer oriented chaperone HSP60, and a 37 cluster of U-shape HSP60 subunit was observed on top of the layered nanotube surface. The ratio of width/length of the HSP cluster from our images is 0.90 vs. 0.88 reported from the literature, our AFM results has a good agreement vs. cryo-EM of 98% [23-25]. FIG. 28D reveals the HSP60 cluster's structure with nanopores of the HSP with double-truncated donuts in shape or U-shape subunits on the top of the orderly nanotube array membrane. FIG. 28E shows another cluster of HSP60 in a 1.4×1.4 μm² area, it emitted golden light beams from the HSP clusters. FIG. 28F shows the nanotube density differently oriented on the top-hill area and on the low-lay canyon area of the 2D AFM image.

### Example 3—Evaluation of the Friedel-Oscillation in the Superlattice Membranes

Friedel-oscillation is a phenomenon of long-range indirect interactions between electrons on a superlattice surface by metal oxide materials [16]. Our group has observed strong Friedel-oscillation events in AFM images based on mono- or multiple-layered organo-metallic materials on SAM surfaces [17-22]. FIG. 29A was with the cysteine “On” in its innate state, and we observed the Friedel-oscillation in the 3D AFM image with an electronic cloud surrounded on the zinc atoms of the toroidal array superlattice. FIG. 29B shows the AFM image having large circulars with zinc created a Josephson junction superconducting qubit device. FIG. 29C depicts an AFM image of the multiple-enzyme network membrane with multiple-cluster high tower structure as “Tesla Tower” having the tower diameters between 500 nm to 2.4 μm, and the towers' height is about 500 nm. The strong Friedel-oscillation from Cooper-pair electron cloud due to the MMP-2. HSP60 networking alignment was observed. We observed many MMP-2 formed toroidal rings with some of them have zinc atoms on top. FIG. 29D depicts such a perfect “wedding ring” with zinc ions mobile from the bottom layer (z value 25.5 nm) to the second layer (z value 86.4 nm) sparkling. Many HSP60's fingerprint structure looks like two-end cut of a pineapple observed along with subunits on the superlattice flat surface. FIG. 29E depicts another AFM image from a different area for the tower glow with Cooper-pair moving with the Friedel-oscillation compared with the background superlattice of toroidal rings. FIG. 29F depicts the 2D AFM image of a HSP60 subunit as a donut at the low z-value 70 nm. FIG. 29G depicts the 3D AFM image of the tall nanometer size “Tesla Tower” with height between 500-700 nm.

FIG. 30A shows an art model for Device 2 of the superconductive/energy sensing device of the HSP60/MMP-2 moonlighting-network in a side view of the Josephson Junction (JJ). “60” is the electrode; “61” is the amplified wave after the Cooper pair went through the multiple superconductor-JJ (the mobile zinc ions from MMP-2)-superconductor (SJJS) layers at a higher frequency. “62” refers to the Cooper pair; “63” refers to the circular current flow in a positive direction with the zinc atoms as the brown balls; “64” refers to the HSP6's double-truncated donut cavity array matrix alignment with each other produced the eternal superconducting current in the blue circle having induced a φ₀, single flux quantum by means of zinc ions bridged from the MMP-2, that a non-ferromagnetic field is produced; “65” is the nano-island membrane on the gold electrode; “66” is the wave of cooper pair electrons after passing through the nano-island membrane; Notice there is an air barrier between the membrane and the array of HSP60 matrix. “67” refers to the PEG . . . PVP's N-terminal chain.

FIG. 30B depicts the detailed art model of the HSP60/MMP-2 with “1” refers to the gold electrode, “2” refers to the MMP-2 toroidal structure comprising of two small toroidal circuits in the center with zinc ions serve as the JJ's “insulator”, “3” reefs to the two pieces of HSP60's truncated double-donuts, “4” refers to the mobile zinc ions from MMP-2 up to the HSP cavity, promoting the long range superconducting current, and “5” refers to the Cooper pair electrons. We conclude that the HSP/MMP device 2 may have superconductive characteristics than that of HSP device 1, due to the observation of the Friedel-oscillation.

### Example 4—Evaluation of the Superconductivity and Memristivity

The hallmarks of the JJ characteristics are (1) at a DC voltage =0,

$${I}_{s} = {I}_{c} sin ( Δφ ) \qquad ( 1 )$$

Iₛ is the supercurrent, I_{c} is critical current, Δφ is the phase difference between the waves of two superconductors appears at the DC Josephson junction; (2) at a finite DC voltage, the phase change of the superconducting wave vs. time caused oscillating at the AC Josephson Junction, and is proportional to 2 eV_{DC}, i.e.,

$$\partial φ / \partial t ∝ 2 e {V}_{D C} \qquad ( 2 ) [ 26 - 28 ]$$

The Josephson junction energy was from the Cooper pair, the magnetic energy was from the inductivity of the circular vortex, and the charge energy was from the SIS quantum capacitor-like device [29]. The vortex suppression of the super current effect also was considered in the equation. However, there was no further analysis of how each component energy contributes to the system superconductivity from the experimental data. Cosmic's group reported seeing the vortex in a Josephson array based on a fractional Josephson Effect in the vortex lattice [30]. The Hamiltonian of the Josephson Junction Array (JJA) was given in the combinations of the first part of charging energy obtained from all arrays and the second part of the Josephson Effect energy [30]. Still, no reports were given on how the energies impacted on one another in their experiment. Inspired by their experimental works, our attempt was, by using the 3D dynamic map method, to further seek a method to elucidate the reactions between the component energies to the superconductivity of the vortex array system at room temperature without external magnetic field applied. Our experimental data were shown on the i-V curves and the AFM structure of the superlattice array. The modified Sine-Gordon system energy for our d-wave vortex array is:

$${E}_{JJA}^{n} = ( 1 / 2 ) {{C}_{i}^{- 1} ( Q - e {n}_{1 … i} )}^{2} \qquad ( 3 )$$

$${E}_{L}^{n} = ( 1 / 2 ) {μ}_{0} {N}_{n = 1 … i}^{2} \cdot A \cdot {L}_{n = 1 … i}^{- 1} \cdot {I}_{n = 1 … i}^{2} \qquad ( 4 )$$

where Eⁿ_{jjA} is the charge energy of Josephson Junction arrays at n=1 . . . i; Q is the charge, C is the total capacitance at n=1 . . . i, en is the n quantum particles at 1 . . . i data point with an energy periodic in h/e for Josephson effect for d-wave [31]; Eⁿ_{L} is the Inductive energy induced by the circular toroidal array. N is the turning number around the toroidal porous at n=1 . . . i, A is the cross-sectional area of the porous, L is the length of the wending, μ₀ is the magnetic permeability constant in free space; I is current. The toroidal arrays are in series connected. Recent publication regarding our FFTJJ mmultiple-variable study results in 3D dynamic maps was presented in the literature [32].

Memristors are devices made of nanolayers that can mimic neuronal synapses with a characteristic of a hysteresis loop in the i-V curve [33-37]. The memristor HSP Sensor 1's hysteretic i-V profiles measured by the CV method are presented in FIG. 31A as control shown in scan rate 20 Hz, 200 Hz, and 1 kHz with the cross-point at zero-bias having zero current, except 10 kHz and 20 kHz lost the memristive. At 20 Hz, the i-V curve shown Aβ alone having two significant oxidative DETₒₓ peaks at 189 mV and 465 mV, but after 40 μg/mL MOX applied in the 250 ng/mL Aβ solution, no DETₒₓ peaks were observed, indicating MOX had impaired HSP60's function. At 1 kHz scan rate for 10 consecutive cycles, the DETₒₓ and DET_{red} peak intensity are the highest at the first scan cycle, it reduced by 30% at the 5^{th} cycle (data not show). In contrast, the MEM_{red} and MEMₒₓ peak intensity showed a “V” shape at the 5^{th} cycle, the lowest, then at the last cycles, the highest, indicates time increases the polarity by memristivity than that of the DET signaling in FIG. 31B, hence the HSP nanopillar vertically oriented on the top of the orderly nanotubes covered of the membrane played a role for the device's function.

In contrast, the HSP60/MMP-2 Sensor 2 shows no DETₒₓ peaks of Aβ in all scan rates. FIG. 32 Panel A at 20 Hz, a large reduction DET_{red} peak at −438 mV was observed, indicates Sensor 2 transferred Aβ from harmful to be useful. At 20 kHz, superconductivity at zero-bias was observed for with or without A3, and with or without MOX in FIG. 32 Panel B. The phase change and super-positioning were also observed [17, 19-22, 26-32]. These observations indicate Sensor 2 expelled Aβ enter HSP's cavity based on its unique toroidal/tower structure.

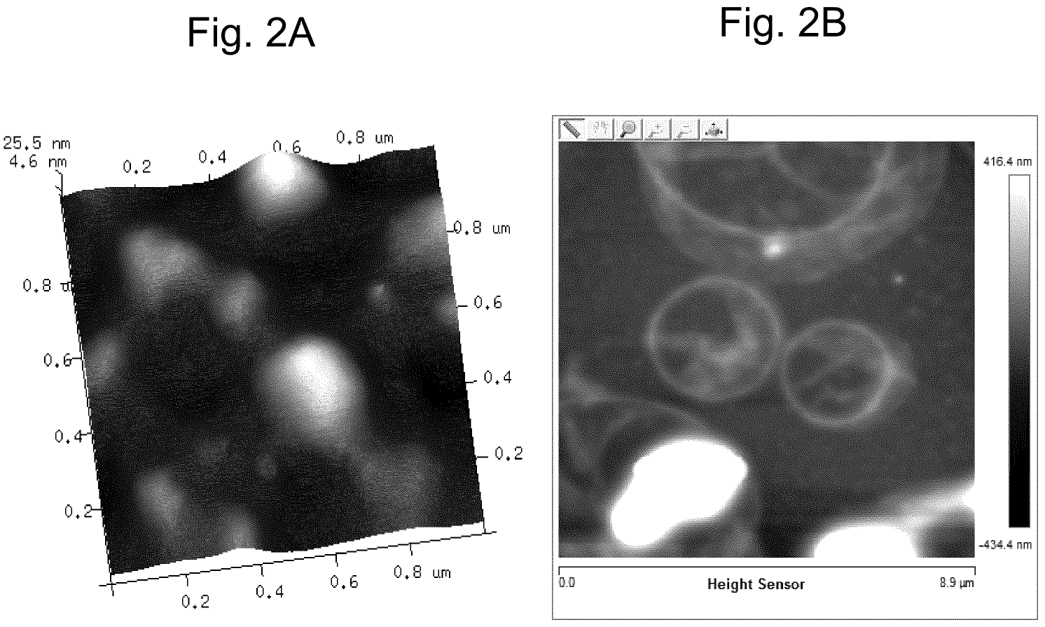

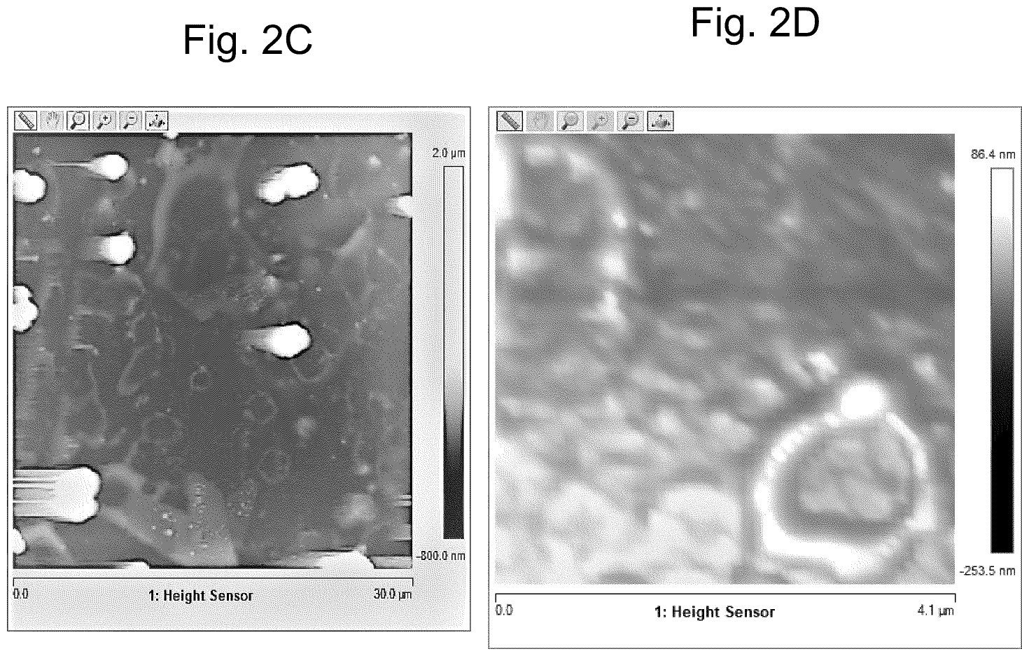

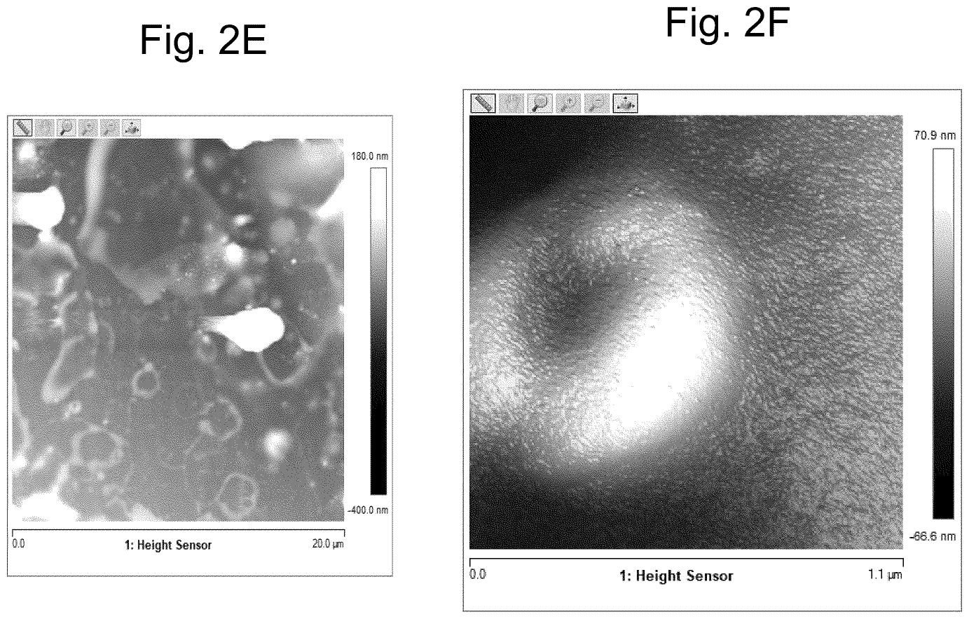

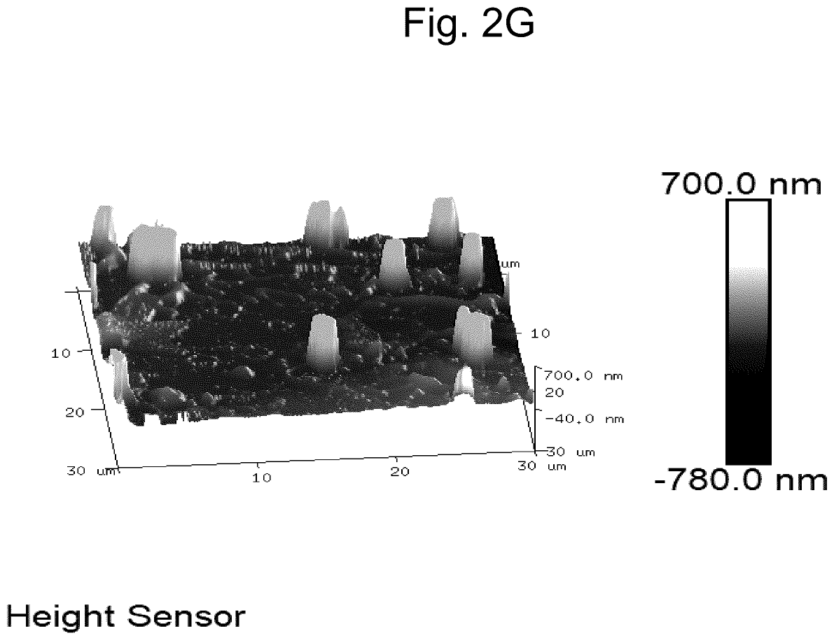

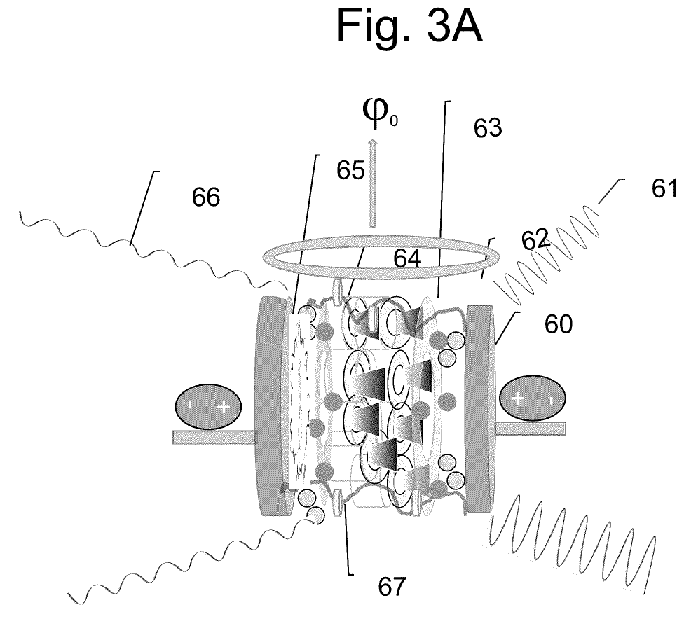

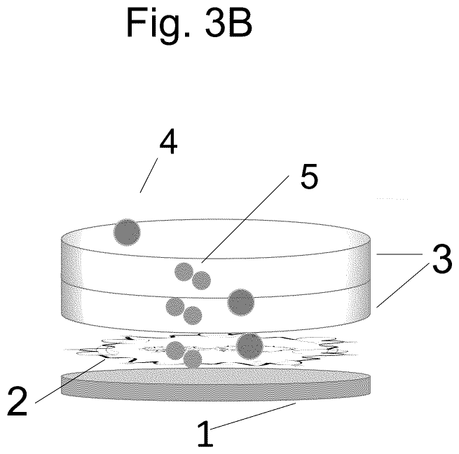

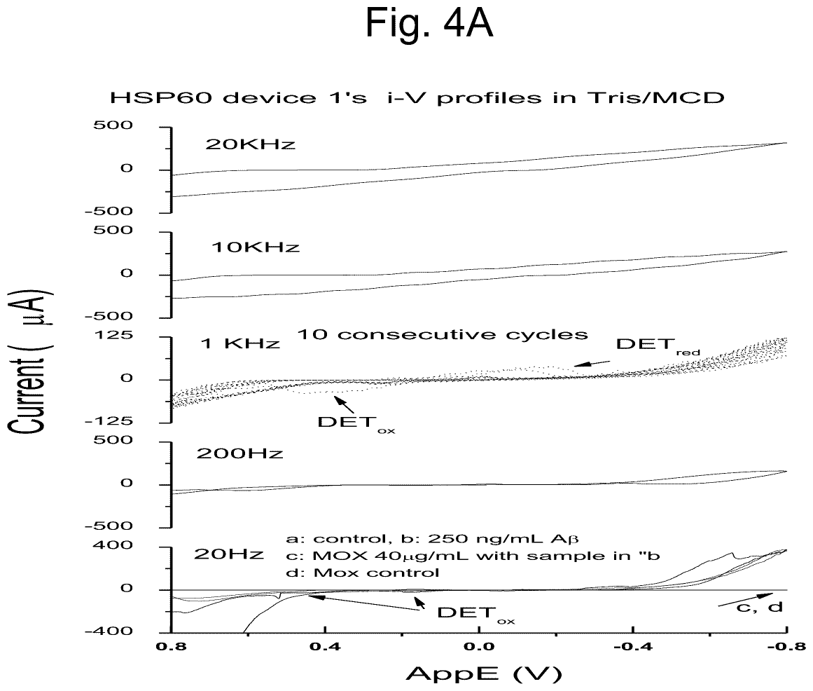

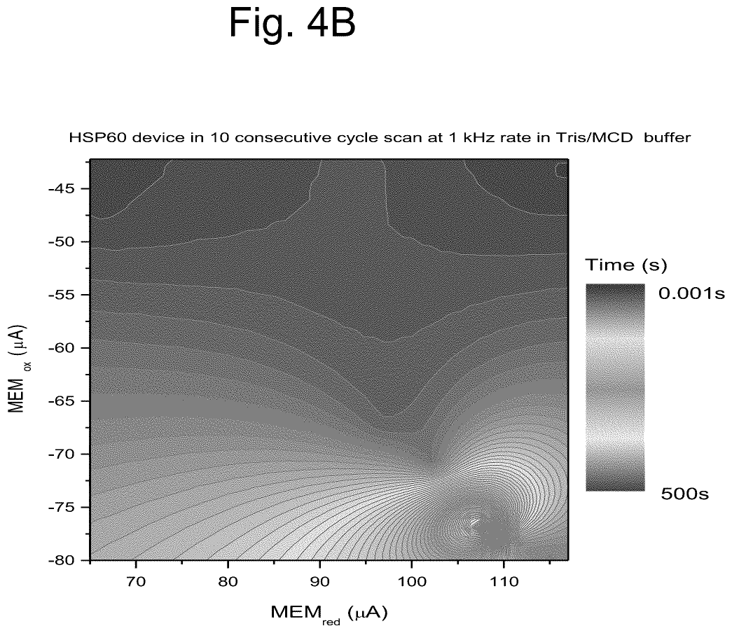

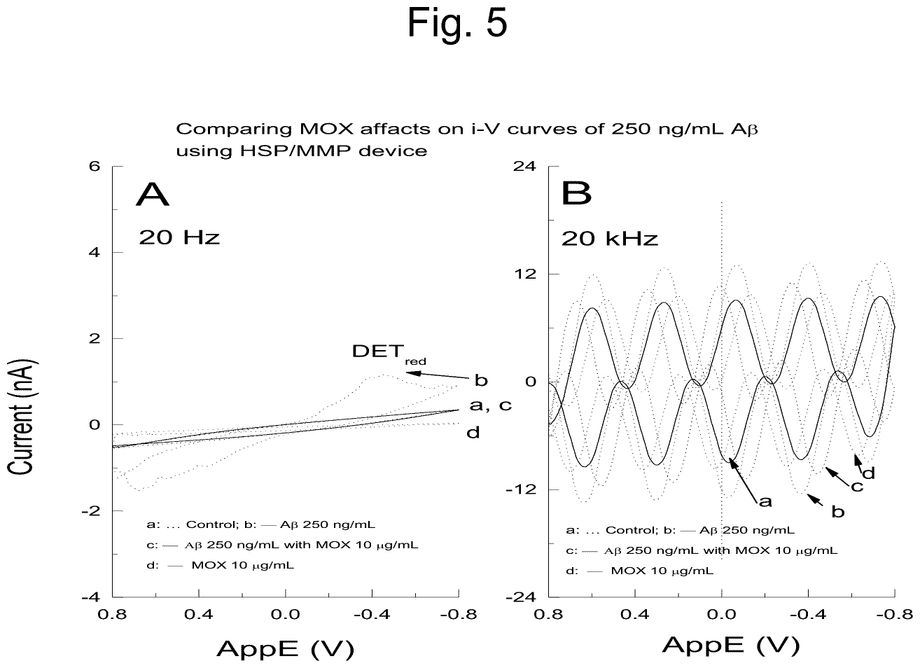

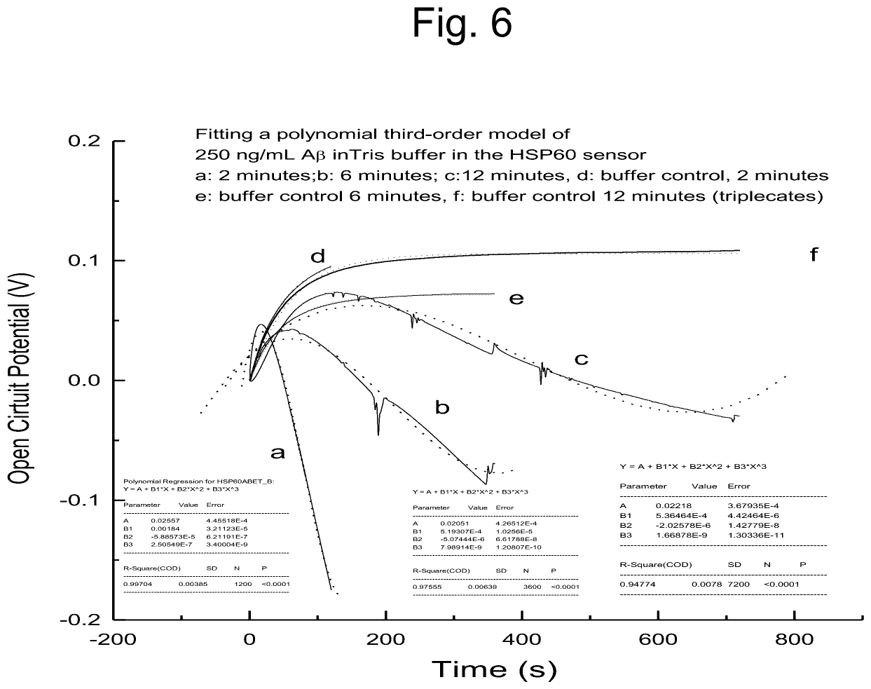

Zinc ions' mobility from MMP-2's superlattice toroidal array layer efflux toward HSP 60's double-ring structured layer and formed a long-range DET relay was demonstrated in FIGS. 2B and 2D. This research confirms clinical doctors' suggestions for adding zinc ionophores for Covid 19 patients' d treatments are sound suggestions that are based on our structural and neuronal circuitry's perspectives [38-39].

### Example 5—Pharmacodynamic Study of Inhibitions of Aβ Protein-Refolding in the HSP or HSP/MMP Cavities Using an OPO Method

The Open Circuit Potential (OPO) of Device 1. Scientists revealed proteins have a funnel-shaped energy landscape with many high-energy, unfolded structures and only a few low-energy, folded structures [40-42]. We expected our devices can be models for assessing protein-folding energy under an open circuit potential (OPO) condition with current=0. Here, the potential vs. time curve results shows in FIG. 33 for Sensor 1 at the 2-, 6-, and 12-minutes monitoring Aβ folding energy landscape for 250 ng/mL Aβ compared with buffer control. Curves with Aβ alone, potential dropped to negative from original equilibrium state (positive potential) was observed, and the data was compared through fitting a polynomial third-order model of y=A+β1*X+β2*X\hat{ }²+β*X\hat{ }³, β1 refers to the coefficient of the linear component, β2, and β3 refer to the coefficient of the curvature, the values of β1 is 0.00184 at 2 minutes monitoring, which is the highest and the β2 has the most negative down dropping power of −5.89e⁻⁵ than at 4 and 12 minutes monitoring, indicates HSP60 alone is vulnerable to toxins' attack. The three buffer control curves have the same first-order rate constant value of 0.98/s based on the exponential curve fitting Box Lucas 1MOD model y=a(1−e^{-bx}), as a result, shown a “healthy” HSP60 sensor in the buffer before Aβ attack. FIG. 34 in Panel A, compared the energy landscape curves at a fixed Aβ concentration with various MOX concentrations at 2 minutes compared to 6 minutes monitoring shown in Panel B, demonstrates MOX's ability to impair Aβ folding completely with rate constants 0.9875/s±0.0047 (4 rate constant values) having an error of 0.76% related to the buffer control rate 0.98/s at 2 minutes monitoring; and a mean rate constant of 0.9877/s±0.0061 (5 rate constant values) with an error of 0.79% related to the buffer control rate 0.98/s at 6 minutes monitoring.

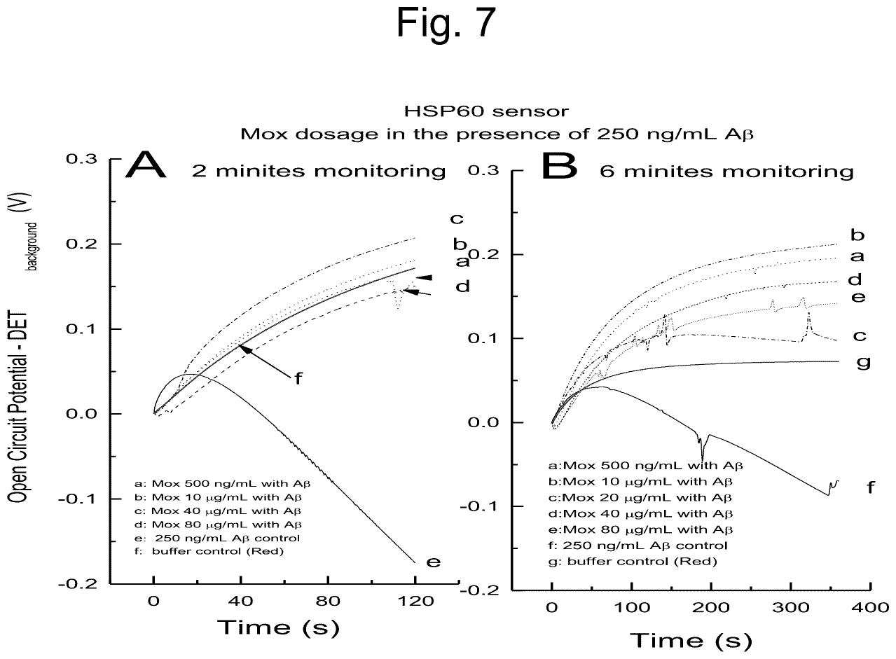

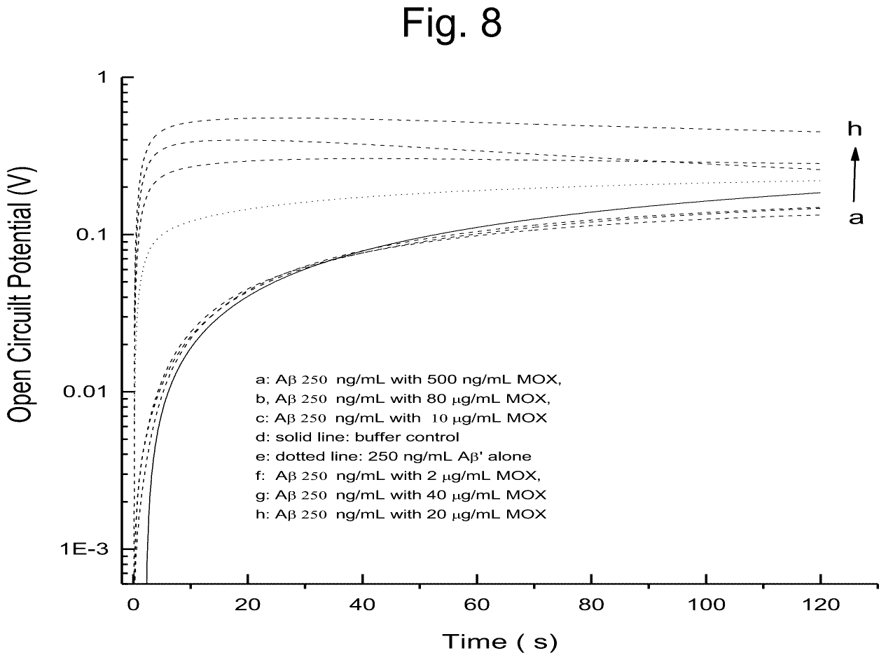

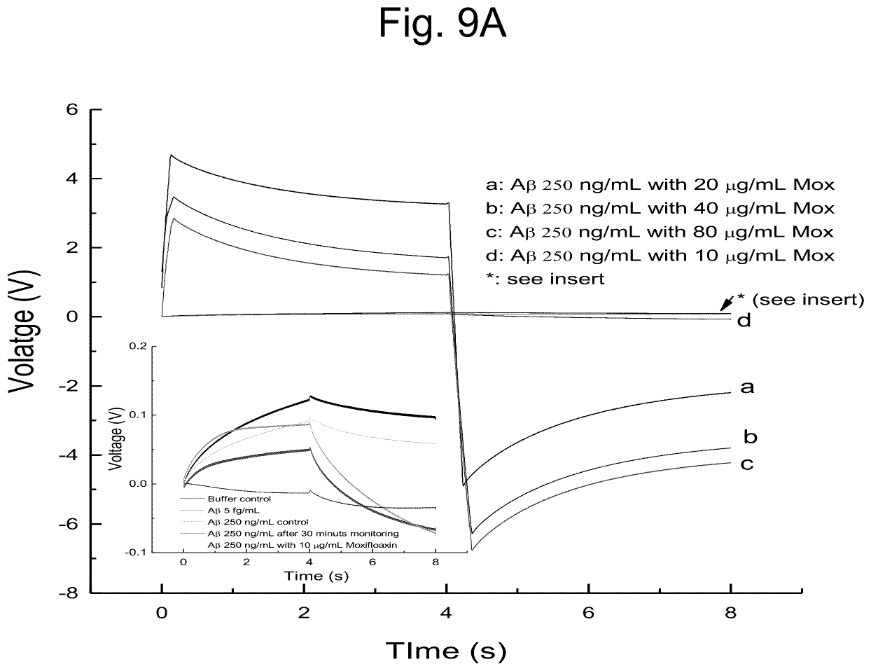

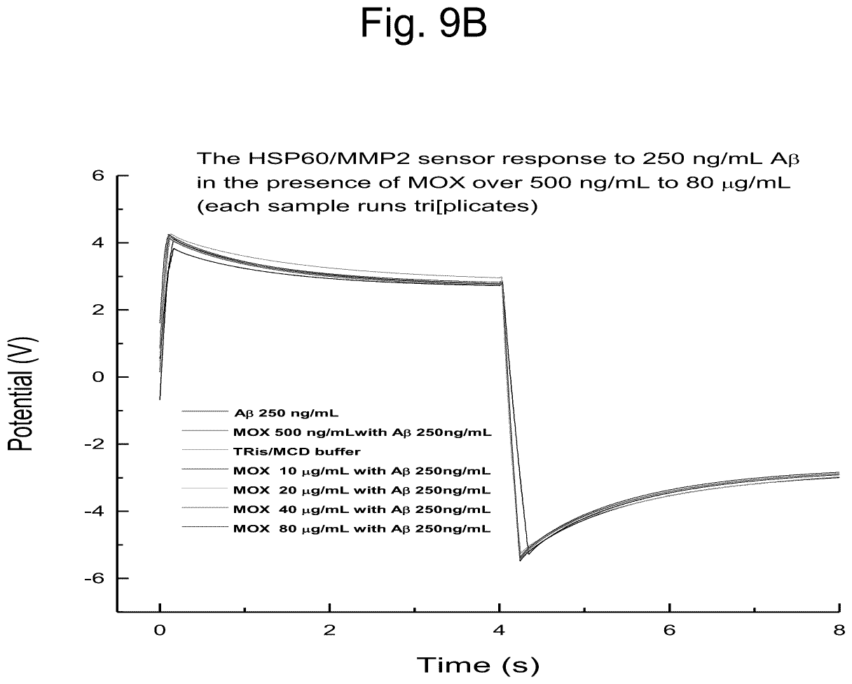

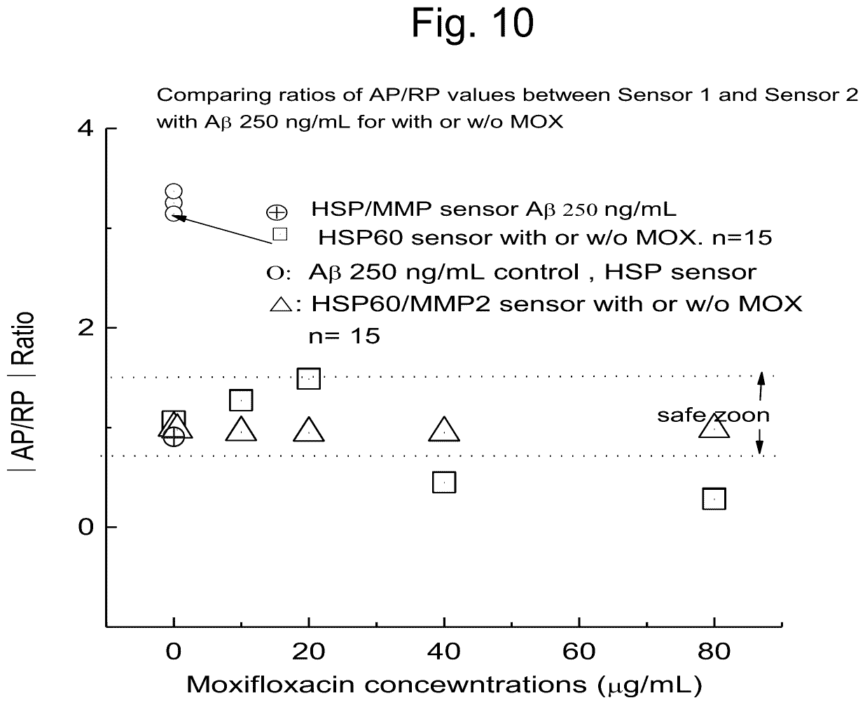

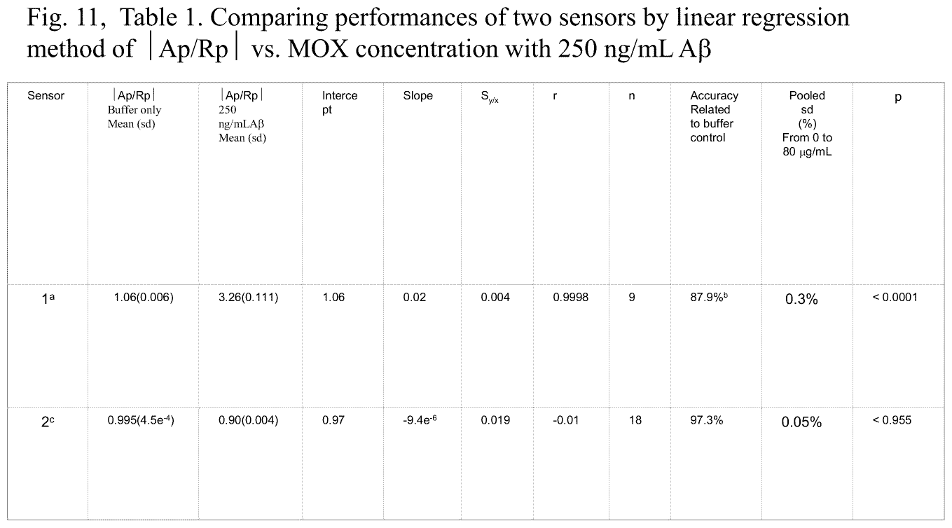

The Open Circuit Potential of Sensor 2. FIG. 35 has the high Aβ concentration that did not cause any oxidative peaks, we further show under different MOX dosages, the potential vs. 2 minutes monitoring curves with the A3, no energy dropped to negative, compared with the buffer control was observed in FIG. 7. The mean rate constant value 0.987 s⁻¹±0.0045 (n=4), produced an imprecision error of 0.16%, and a good agreement with the control's rate constant was obtained in 99.8%, except MOX at 0.5 μg/mL and 80 μg/mL, because curves drifting occurred and were failed to fit the model.

### Example 6—Recovery of the Reversible Membrane Potential (RMP) Through Moxifloxacin

We first reported using a ratio of action/resting potential to monitor biomarkers of diseases [17-18], because keeping a normal RMP is essential for maintaining healthy cells. Moxifloxacin changed the energy profiles of Sensor 1 as shown in FIG. 36A, that the voltage vs. time curves changed from asymmetric to symmetric when MOX concentration increases from 10 mg/mL to 80 mg/mL in the presence of 250 ng/mL Aβ vs. the controls. FIG. 36B depicts Device 2's profiles with MOX from 0.5 to 80 μg/mL (5 levels), no asymmetric curves were observed under the same experimental conditions. The ratio of results is presented in FIG. 37 compared with the healthy ratio standard. The MOX concentrations are safe in the range up to 20 μg/mL(n=9) in maintaining RMP, and 80 μg/mL is too far from the safe zoon when A3 250 ng/mL for Sensor 1. The 40 μg/mL MOX is out of safe zoon by 35.0%±0.2%. In contrast, Sensor 2's ratio values are all located in the safe zoon up to 80 μg/mL MOX (n=18) compared with controls, indicates the HSP60/MMP2 moonlighting network enhanced the health states of cells' RPM by MOX inhibiting HSP's chaperoning function at a clinically harmful level Aβ.

Comparing performances of two devices by linear regression method of |Ap/Rp| vs. MOX concentration with 250 ng/mL Aβ of a severe stage Alzheimer's. From results of L-S regression in FIG. 38 of Table 1, we conclude MOX promoted RMP for both sensors, only Sensor 1 shows a dependency of the Ap/Rp ratio values on the MOX concentration up to 20 μg/mL located in the safety zoon (n=9, p<0.0001). Including the data in 40 and 80 μg/mL, an accuracy of 87.9% (n=15, 5 levels) produced related to the ratio of buffer samples, and a related pooled standard division of 0.3% was reached. Sensor 2 produced 97.3% accuracy result with a MOX range up to 80 μg/mL (n=18), further shows the ratio values are independent on MOX concentrations with an R-value of −0.01 and a slope −9.4e⁻⁴ and p<0.955, that demonstrated a normal RPM is accomplishable over a wide MOX concentrations in the middle of an Aβ attacking.

### Example 7—Conclusions

The two sensor models for assessing protein refolding energy landscape and evaluations of antibiotic drug impact on the refolding are accomplished by direct real-time monitoring the intrinsic equilibrium energy using the OPO approach through innovations of fabricating nanostructured HSP60 and HSP60/MMP-2 polymer cross-linked SAMs on the electrodes. The results produced correlated well with the evaluation of the RPM effect. Sensor 2 demonstrated the ability in maintaining of normal RPM with a good result of accuracy, and it was not depending on MOX concentrations. The discovery further confirms the moonlighting innate HSP60/MMP-2 network proteins utilized the toroidal array/tower nanostructure and the zinc ions' efflux effect enabled the tunnelling Josephson junctions extended to the HSP cavity. The technology may find therapeutic applications in the future.

### REFERENCE

1. P. G. Jobin, G. S. Butler, C. M. Overall, New intracellular activities of matrix metalloproteinases shine in the moonlight, BBA Molecular Cell Research, 2017, 1864, 2043-2055.
2. E. A. Taha, K. Ono and T. Eguchi, Roles of Extracellular HSPs as Biomarkers in Immune Surveillance and Immune Evasion, Int. J. Mol. Sci. 2019, 20, 4588.
3. G. Mohammad and R. A. Kowluru, Novel Role of Mitochondrial Matrix Metalloproteinase-2 in the Development of Diabetic Retinopathy, Invest Ophthalmol Vis Sci. 2011; 52:3832-3841.
4. A. B. Uzdensky, Multifunctional proteins, Molecular Biophysics, 2020, 65, 390-403.
5. B. Henderson et al., (eds), Protein moonlighting in biology and medicine, John Wiley & Sons, Inc., Hoboken, New Jersey, 2017.
6. M. Bakic, S. Balusu, C. Libert, and R. E. Vandenbroucke, Friends or Foes: Matrix Metalloproteinases and Their Multifaceted Roles in Neurodegenerative Diseases, Mediators of Inflammation, 2015, article ID 620581, page 1-27.
7. T. Zininga, L. Ramatsui and A. Shonhai, Heat shock proteins as immunomodulants, Molecules 2018, 23, 2846, doi:10.3390/molecules23112846.
8. S. Sayad and S. Sayad, Cyclosporin A is a potential inhibitor of SARS-CoV-2. Pre published on March 2020 at publication of Research gate of 340037699.
9. G. Kaul and H. Thippeswamy, Role of Heat Shock Proteins in Diseases and Their Therapeutic Potential, Indian J Microbiol, 2011, 51(2):124-131.
10. A. A. A. Asea, N. N. Almasoud, S. Krishnan, P. Kaur, (Editors), Heat Shock Protein-based Therapies, Springer Chain Heidelberg New York. 2015.
11. K. Marciniec, A. Beberok, P. Pȩcak, S. Boryczka, D. Wrześniok, Ciprofoxacin and moxifloxacin could interact with SARS-CoV-2 protease: preliminary in silico analysis, Pharmacological Reports, 2020, 72:1553-1561.
12. I. Karampela and M. Dalamaga, Could respiratory fluoroquinolones, levofloxacin and rnoxifloxacin, prove to be beneficial as an adjunct treatment in COVID-19? Arch Med Res. 2020, 51(7), 741-742a
13. H. Han, Q. Luo, F. Mo, L. Long, W. Zheng, SARS-CoV-2 RNA more readily detected in induced sputum than in throat swabs of convalescent COVID-19 patients, The Lancet Infections 2020, 20, 655-656.
14. J. A. Balfour, H. M. Lamb, Moxifloxacin: a review of its clinical potential in the management of community-acquired respiratory tract infections, Drugs, 2000, 59(1):115-39.
15. E. T. Chen, J. T. Thornton, S-H. Duh, Topological Superconductive and Memristive Nanostructured Toroidal-Tower Array Device System Enabled Cooper-Pairs Reentry Between States for Sensing Multiple Biomarkers, Sensors & Transducers, 2020, 243(4), 6-23.
16. M. Ternes, M. Pivetta, F. Patthey, and W-D Schneider, Creation, electronic properties, disorder, and melting of two-dimensional surface-state-mediated adatom, Progress in Surface Science 2010, 85, 1-27.
17. E. T. Chen, Chapter 3 of Room-Temperature Self-Powered Nanostructure Organo-Metallic Superconductive Devices in the book of Nanoengineering, Quantum Science, and, Nanotechnology Handbook (Editor S. E. Lyshevski), CRC Press, ISBN Number: 978-0-367-19751-3 (Hardback), Dec. 2, 2019.
18. E. T. Chen, J. T. Thornton and S-H. Duh, 3D-Cage Structured Biomimetic MMP-2/HSP60-Like Organo-Metallic Devices Sensitively Response to ATP Concentration Change Used for Defining the Anti-inflammatory and Pro-inflammatory Status, Sensors & Transducers, 2019, 236 (8), 28-39.
19. E. T. Chen, J. T. Thornton, P. T. Kissinger, and S-H. Duh, Nanostructure Protein Toroidal Array Devices Promoted Room Temperature Superconductivity and Direct Sensing of Collagen-1, Sensors and Transducers 2018, 228(12), 48-62.
20. E. T. Chen, J. T. Thornton, P. T. Kissinger and S-H. Duh, Utilization of the Flexible Fractional Josephson Toroidal Arrays for Sensing, Memory Storage and Quantum Computing, Sensors and Transducers 2018, 228(12), 30-47.
21. E. T. Chen, J. T. Thornton, P. T. Kissinger and S-H. Duh, Nanobiomimetic structured superconductive/memristive organo-metal devices at room temperature serve as amperometric sensors for sub fg/mL Collagen-1 detection, TechConnect Briefs in Biotech, Biomaterials and Biomedical, 2018,106-109.
22. E. T. Chen, J. T. Thornton, P. T. Kissinger and S-H. Duh, Discovering Of Collagen-i's Role in Producing Superconducting Current in Nanobiomimetic Superlattice Structured Organometallic Devices at Room Temperature Enabled Direct Quantitation of Sub pg/mL Collagen-1, TechConnect Briefs in Informatics, Electronics, and Microsystems, 2018, 43-46.
23. Y. Gomez-Liorente, F. Jebara, M. Patra, et. al., Structure basis for active single and double ring complexes in human mitochondria HSP60-HSP10 chaperonin, Nature Communications, 2020, 11:1916. doi.org/10.1038/s41467-020-15698-8.
24. J. Wang, A. S. Enriquez, J. Li, A. P. B. Sigler, Z. Xu, H. S. Rye, S. G. Burston, et. al., Structure and function in GroEL-mediated protein folding, Annu. Rev. Biochem. 1998, 581-608.
25. Rodriguez, et. al., MitCHAP-60 and hereditary spastic paraplegia SPG-13 arise from an inactive hsp60 chaperonin that fails to fold the ATP synthase β-subunit, Scientific Reports, 2019, 9: 12300. doi.org/10.1038/s41598-09-48762-5.
26. Editors E. Wolf, G. Arnold, M. Gurvitch and J. Zasadzinski, Josephson Junctions, History, Devices, and Applications, Pan Stanford Publishing Pte, Ltd, 2017.
27. M. J. A. Stoutimore, A. N. Rossolenko, V. V. Bolginov et al., Second-harmonic current-phase relation in Josephson junctions with ferromagnetic barriers, Phys. Rev. Lett. 2018, 121, 177702.
28. S. Lederei, Y. Schattner, E. Berg, & S. A. Kivelson, Superconductivity and non-Fermi liquid behavior near a nematic quantum critical point. PNAS, 2017, 114(19), 4905-0-10.
29. E. Grosfeld, and A. Stern, Observing Majorana Bound States of Josephson Vortices in Topological Superconductors, PNAS, 2011, 108(29), 11810-11814.
30. R. Cosmic, H. Ikegami, A. Lin, and K. Inomata, Circuit QED-based Measurement of Vortex Lattice Order in a Josephson Junction Array, Physical Review B, 2018, 98 (6), 060501-060505.
31. F. Loder, A. P. Kampf, T. Kopp, et al., Magnetic Flux Periodicity of h/e in Superconducting Loops, Nature Physics, 2008, 4, 112-115.
32. E. T. Chen, J. T. Thornton, P. T. Kissinger, and S-H. Duh, Utilization of the Flexible Fractional Josephson Toroidal Arrays for Sensing, Memory Storage and Quantum Computing, Sensors and Transducers, 2018, 228(12), 30-47.
33. D. Biolek, M. D. Ventra and Y. V. Pershin, Reliable SPICE Simulations of Memristors, Memcapacitors and Meminductors, Radioengineering 2013, 22 (4), 945-968.
34. J. Martinez-Rincon and Y. V. Pershin, Electron Devices, IEEE Transactions 2011, 8(6), 1809-1812.
35. J. Martinez-Rincon, M. D. Ventra, Y. V. Pershin, Solid-State Memcapacitive System with Negative and Diverging Capacitance, Physical Review B, 2010, 81(19), 195430-1-195430-7.
36. M. D. Pickett, G. Medeiros-Ribeiro, and R. S. Williams, A Nature Materials, DOI: 10.1038/NMAT3510, 2012.
37 Kozma R, Pino R E, Pazienza G E, Advances in neuromorphic memristor science and applications, Springer publisher, 2012.
38. N. Samad, T. E. Sodunke, A. R. Abubakar, et al., The implication of zinc therapy in combating the COVID-19 global pandemic, J. Inflammation Research, 2021, 14, 527-550.
39. A. Mayor-Ibarguren, C. Busca-Arenzana, and A. Robles-Marhuenda, A hypothesis for the possible of zinc in the immunological pathways related to COVID-19 infection, Frontiers in Innunology, 2020, July 10. doi.org/10.3389/fimmu.2020.01736.
40. I. Wessels, B. Rolles, and L. Rink, The potential impact of zinc supplementation on COVID-pathogenesis, Frontiers in Immunology, doi.org/10.3389/jimmu.2020.01712.
41. I. Sorokina and A. Mushegian, Modeling protein folding in vivo, Biology Direct, 2018, 13, 13.
42. B. Schiffrin, D. J. Brockwell and S. E. Radford, Outer membrane protein folding from an energy landscape perspective, BMC Biology, 2017, 15, 123
43. K. A. Dill and J. L. MacCallum, The Protein-Folding Problem, 50 Years On Science 2012, 338, 1042.
44. P. C. Ke, R. Zhou, L. C. Serpell, R. Riek et. al., Half a century of amyloids: past, present and future, Chem. Soc. Rev., 2020, 49, 5473.

### Detailed Descriptions of the CIP Invention

### Example-1. Fabrication of the Double-Layer Nanostructured Josephson Junction Toroidal Array Superlattice Biomimetic ACE2 Sensor

Device 1 was freshly prepared with two steps: first, by the self-assembling method with compositions of triacetyl-β-cyclodextrin (TCD), polyethylene glycol diglycidyl ether (PEG), poly(4-vinylpyridine) (PVP) and β-cyclodextrin copolymer (β-CD) as a mixture with appropriate proportions and forming nano island layer 1 that mimics choline acetyltransferase (CHAT) on a 50 nm gold chip at 35° C. for 48 hrs. Second, after a washing and drying process, we deposit the second polymer mixture of bis-substituted dimethyl-β-cyclodextrin (bM-β-DMCD)/TCD/PEG/PVP and embed it with zinc chloride on the top of the first layer. For the first 2 hours, the temperature was kept at 80° C. After that, the temperature was reduced to 37° C. for 96 hours. Other washing and dry procedures we used were based on the literature [13]. The compositions and volume ratios of the compositions in the nano-island membrane of TCD/PEG/PVP/CD Copolymer were disclosed in U.S. Pat. No. 8,632,925 Jan. 21, 2014, and U.S. Pat. No. 9,793,503, Oct. 17, 2017). The second polymer mixture has a volume ratio 40-60%:10-20%:8-10%:8-10%:7-15% for bM-β-DMCD/TCD/PEG/PVP/ZnCI₂, respectively, was disclosed in U.S. Pat. No. 11,531,924, 2022. The concentration of bM-β-DMCD is in the range of 5-10 mg/mL in HEPES. The first four components were incubated for 2 hours, and then apply the zinc chloride solution into the mixture. For the first 2 hours, the temperature was kept at 80° C. Procedures of synthesis and characterization of mM-β-DMCD and bM-β-DMCD were based on the published literature [14].

### Example-2 Fabrication of the Native Protein ACE2 Sensor with Memristive Characteristics

The Fabrication of the native protein ACE2 sensor was conducted according to a procedure published in literature [15]. In brief, the Self-Assembled Membrane (SAM) was fabricated by a mixture solution of copolymers cross-linked with triacetyl-β-cyclodextrin (TCD), polyethylene glycol diglycidyl ether (PEG) and poly(4-vinylpyridine) (PVP) and ACE2 with an appropriate composition deposited on a gold chip with Ag/AgCl as the reference electrode, and incubated for 72 hours at 37° C. The ACE2 concentration embedded was 230 nM. The morphology of the AU/SAM was characterized using an Atomic Force Microscope (AFM) (Asylum).

### Example-3 Synthesis of the ABS02 Inhibitor for S1 SARS-CoV2 Virus

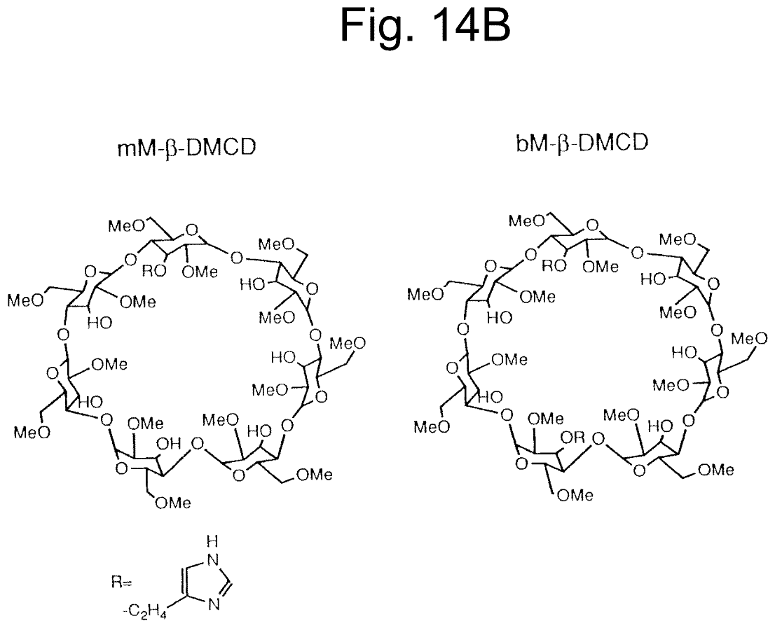

Synthesis of the green color bis imidazole groups modified β-dimethyl cyclodextrin (0-DMCD), namely bM-β-DMCD in the cavity of cyclodextrin was conducted by the published literature [14]. The molecular formula is C₆₆H₁₁₀O₃₅N₄·18H₂O with a MW=1519.6+18H₂O=1843.6 for bM-β-DMCD. The chemical name of bM-β-DMCD is: C-3,3′-(bis[2-(4-imidazolyl)ethyl]-dimethyl-β-cyclodextrin. The chemical structure is shown in FIG. 14B on the right-side structure. Synthesis of the yellow color mono-imidazole groups modified β-dimethyl cyclodextrin (β-DMCD), namely mM-β-DMCD in the cavity of cyclodextrin was conducted by the published literature [14]. The chemical name of mM-β-DMCD is: C-3-(2-(4-imidazolyl)ethyl]-dimethyl-β-cyclodextrin. The molecular formula is C₆₁H₁₀₄O₃₅N₄·4H₂O with a MW=1425+4H₂0=1497 for mM-β-DMCD. The chemical structure of mM-β-DMCD is shown in FIG. 14B on the left-side structure. The ABS02 inhibitor comprises of (1) bM-β-DMCD 6 mg/mL, (2) triacetyl-β-cyclodextrin 5-6 mg/mL (TCD), (3) polyethylene glycol diglycidyl ether (PEG) 0.3-0.5 mg/mL, and (4) poly (4-vinyl pyridine) (PVP) 0.4-0.6 mg/mL, each component has a 6-10% (V/V) of the total mixture, except component 1 has a 60-65% (V/V). Mix the mixture well in degassing and sonicating, then incubated at 37° C. for 2 hours, after that, add (5) ZnCl₂ 2.5 mM 8% (V/V) of 1-4, and (6) cysteine 1.2 mM 8% (V/V), incubate for another 2 hours at the same temperature. When finished, add (7) 8-10 mg/mL mM-β-DMCD in 22% (V/V) and (8) 7-10% (V/V) collagen-1 in the total mixture, further incubate for another 2 hours in the same temperature. The measurement of the sample size is 20 μL for validation. The standard S1 inhibitor Remdemivir, was approved by US FDA, was purchased from Sigma, and the S1 (1-681, MW76 kDa, recombined) antigen of SARS-CoV-2 was heat inactive and was purchased from Axxora, NY11735.

### Example-4 Evaluation of the Friedel-Oscillation in the Superlattice Membrane of the Biomimetic ACE2 Sensor

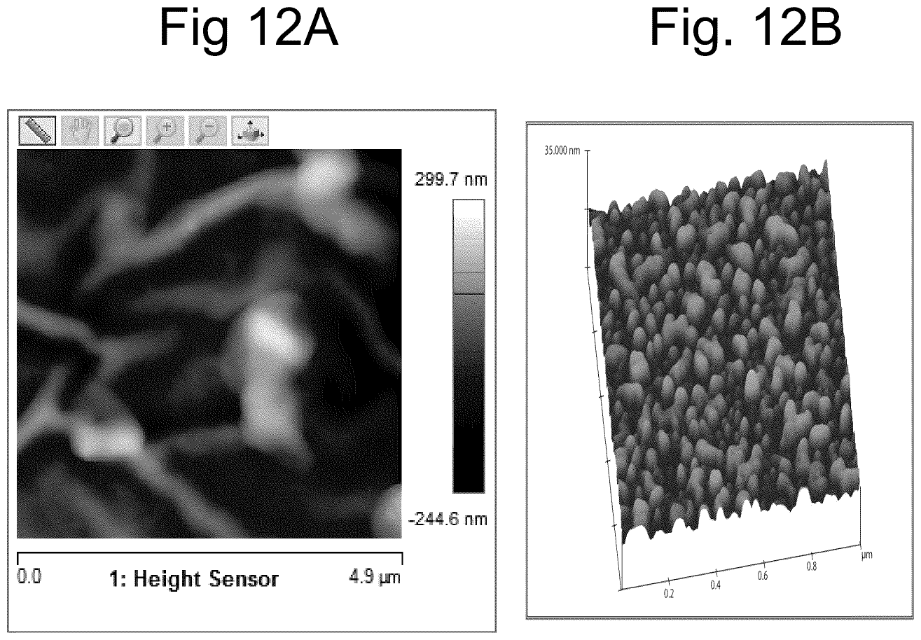

The morphology of the AU/SAM was characterized using an Atomic Force Microscope (AFM) (model Dimension Edge AFM, Bruker, MA). Data was collected in TappingMode using silicon probes with a 5-10 nm tip radius and ˜300 kHz resonance frequency (Probe mode TESPA-V2, Bruker, MA). Evaluations of the Friedel-oscillation on the qubit device membrane were conducted based on the AFM images. Friedel-oscillation is a phenomenon of long-range indirect interactions among Cooper-pairs electrons moving toward same direction on the surface, that might show the potential application of the surface materials used for superconductive purposes [16]. The bird view of the AFM image presented here is for the double-layer SAM membrane of Sensor 1 as depicted in FIG. 12A with curvature single-wall nanotube with zinc atoms with strong Friedel-oscillation observed around the zinc cluster atoms; the highest z is 299.7 nm on 24.2 μm² with surface roughness 77.5 nm related to z-direction of 435 nm, the zinc atoms play a role as an insulator or “bridge”, acts as the Josephson junction in an orderly manner connecting curvature nanotubes in the superlattice in FIG. 12A. It was clearly demonstrated the toroidal JJ array qubits in an orderly matrix with zinc Josephson junctions. FIG. 12B depicts the first layer of the 2D AFM image of the SAM organic conductive membrane that mimicked CHAT function in the nano-island structure in 1.0 μm2.

### Example—5 Evaluation of the Native Protein ACE2 Surface SAM Membrane Structures

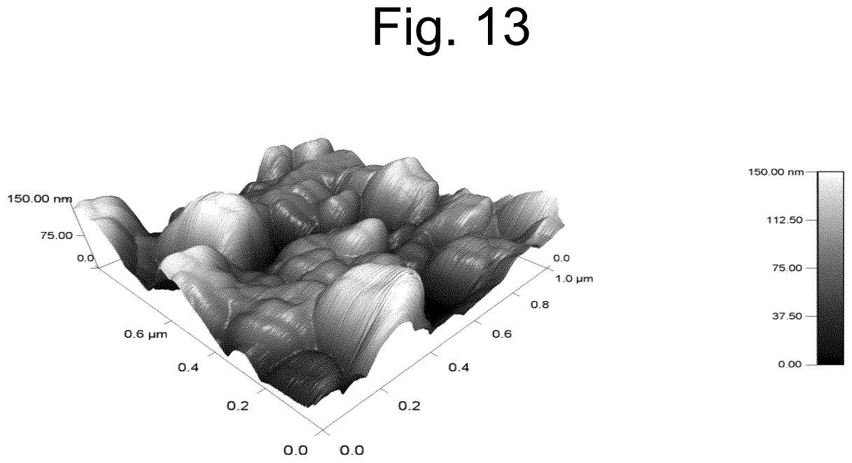

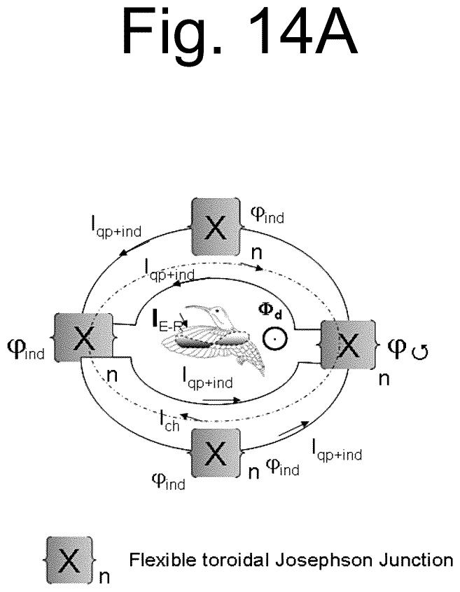

FIG. 13 depicts the 3D AFM image of the Native Protein ACE2 Surface SAM Membrane in the presence of ACE2 from 230 nM cross-linked with conductive polymers in a 1 μm² surface view with a Z direction height of 150 nm. The surface is not smooth containing of several large ACE2 bombs compared with the lower ACE2 contain SAMs in 57.5 nM and 4.5 nM ACE2, respectively [15]. ACE2 is a multiple function enzyme comprising of a C-terminal region (carboxy domain), N-terminal peptide region and an HEXXH zinc finger metalloprotease motif (catalytic domain) [17]. Paul Towler's group's x-ray structure revealed the native ACE2 formed a zinc finger comprising of His³⁷⁴, His³⁷⁸ Glu⁴⁰², one water molecule, and coordinated with Zn⁺² at the active site [18]. Sensor 2 shows the high surface roughness with more nanostructure bumps, and small balls in the membrane in FIG. 13. Our work used ACE2 cross-linked with triacetyl-fi-cyclodextrin (TCD), polyethylene glycol diglycidyl ether (PEG) and poly(4-vinylpyridine) (PVP) has enriched the zinc-finger from ACE2 and associated with the catalytic subdomain α-helix from the PEG and the PVP, and the COO⁻ of the TCD further enriches Glu⁴⁰²'s function evidenced by the increased ACE2 in the SAMs showed a transformed surface with densely packed large nano-bumps and small nano-balls.

### Example—6 Flexible Josephson Toroidal Junction Array

FIG. 14A depicts the circuitry symbol of the biomimetic ACE2 sensor with Flexible Toroidal Josephson Junction Superlattice Quantum Qubit (FTJJSLQUBIT) arrays comprising of at least 4 or more junctions and a self-powered switchable reversible electron-relay in the center which is the fundamental function of the biomimetic ACE2 self-assembled membrane (SAM) that the mems-element construction relies on. The junction materials are various and can comprise the dielectric insulator, and transitional metal atoms, such as zinc atoms; or inert material, such as air. The FTJJ is a component of a circuit that has the mems-inductor and memristor connected in parallel that connected with a mem-capacitor in serial position that produces a circuit having functions once the switchable reversible electron-relay current produces an open circuit potential enough to self-powered the chip circuit, herein such as memory storage, operation, and energy storage in the same device without a need of a microwave power supply. The hummingbird's “8” shape fly pattern is a symbolic representation of the intrinsic electron-relay loop within and between the membranes that initiated the cooper pair tunneling and crossed the JJ barriers.

The modified Sine-Gordon system energy for our d-wave vortex array is:

$${E}_{JJA}^{n} = ( 1 / 2 ) {{C}_{i}^{- 1} ( Q - e {n}_{1 … i} )}^{2} \qquad ( 1 )$$

$${E}_{L}^{n} = ( 1 / 2 ) {μ}_{0} {N}_{n = 1 … i}^{2} \cdot A \cdot {L}_{n = 1 … i}^{- 1} \cdot {I}_{n = 1 … i}^{2} \qquad ( 2 )$$

where E^{a}_{jjA} is the charge energy of Josephson Junction arrays at n=1 . . . i; Q is the charge, C is the total capacitance at n=1 . . . i, en is the n quantum particles at 1 . . . i data point with an energy periodic in h/e for Josephson effect for d-wave [19]; Eⁿ_{L} is the Inductive energy induced by the circular toroidal array. N is the turning number around the toroidal porous at n=1 . . . i, A is the cross-sectional area of the porous, L is the length of the wending, μ₀ is the magnetic permeability constant in free space; I is current. The toroidal arrays are in series connected. Recent publication regarding our FFTJJ mmultiple-variable study results in 3D dynamic maps was presented in the literature [20]. The RF-SQUID consists of a superconducting ring of inductance L interrupted by a JJ, the potential energy of the SQUID and the Hamiltonian equations are given by:

$$U ( Φ ) = {( Φ - {Φ}_{e} )}^{2} / 2 L - {E}_{J} cos ( 2 πΦ / {Φ}_{0} ) \qquad ( 3 ) [ 21 ]$$

$$H = {Q}^{2} / 2 C + {( Φ - {Φ}_{e} )}^{2} / 2 L - {E}_{J} cos ( 2 πΦ / {Φ}_{0} ) \qquad ( 4 ) [ 21 ]$$

Φₑ is the applied magnetic flux penetrating the SQUID ring.
Φ is the total magnetic flux threading the SQUID ring.
L is the inductance.
Eᵢ represents the Josephson coupling energy.
Φ₀ is the superconducting magnetic flux quantum.
Q is the charge on junction's shunt capacitance satisfying [Φ, Q]=ih/2π
h is the Planck constant.

### Example-7 Molecular Structures of the S1 SARS-CoV2 Inhibitors

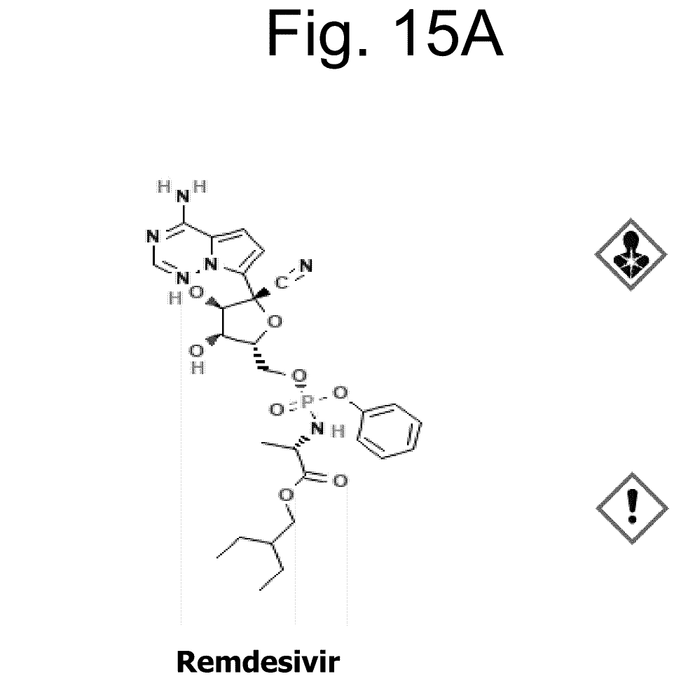

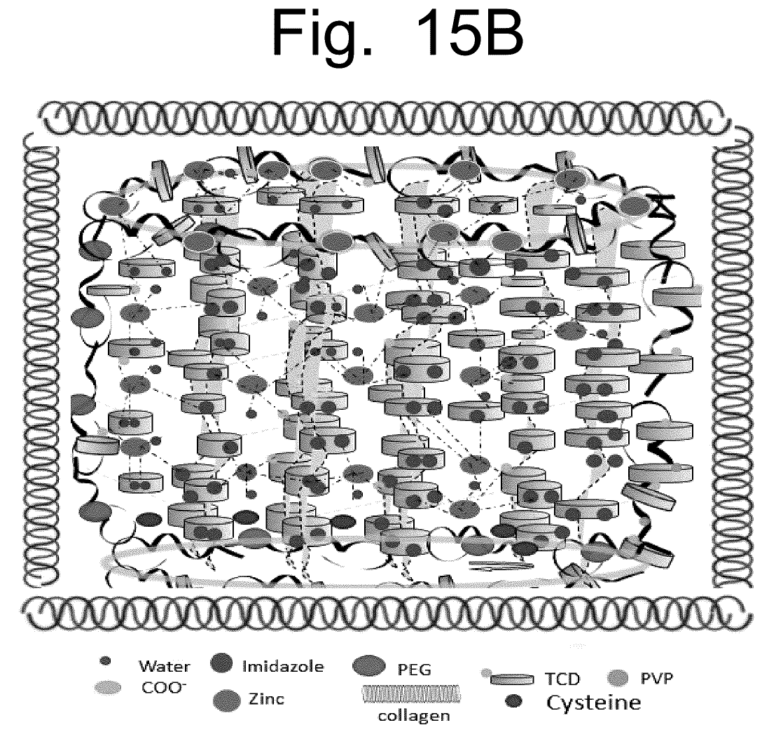

FIG. 15A depicts the molecular structure of the US FDA-approved S1 SARS-CoV2 virus inhibitor drug Remdesivir (REM). REM is a nucleotide prodrug of an adenosine analog. It binds to the viral RNA-dependent RNA polymerase and inhibits viral replication by terminating RNA transcription prematurely. Remdesivir has demonstrated in vitro and in vivo activity against SARS-CoV-2 [22]. The chemical itself has a toxic impact on human organs as cited in the Sigma-Aldrich website.

FIG. 15B depicts the molecular structure of another S1 SARS-CoV2 virus inhibitor, namely Advanced Biomimetic Sensor S1 inhibitor (ABS02) invented by ABS company. ABS02 is a 3D cage-structured inhibitor comprising supramolecules of bM-β-DMCD, mM-β-DMCD, TCD, PEG, PVP, cysteine, zinc chloride, and collagen cross-linked. ABS02 is an inhibitor to S1 SARS-CoV2 virus, and has stronger biological communication with the S1 Cov2 virus compared with ACE2 using Sensor 1, and using Sensor 2. For the detailed evidence is provided in the following Sections.

FIG. 15C depicts a scheme of Sensor 1 detects S1 SARS-CoV2 virus with or without ABS02 S1 inhibitor in three steps: Step 1 depicts Senso1′'a nanostructured membrane forms the Direct Electron Relay (DET) with a hemming bird-like hysteresis current loop when a voltage is applied and scanned. Step 2 depicts the membrane forms a chelating complex when the S1 virus particle. Step 3 depicts S1 virus selectively interacts with the inhibiter forms a long range DET relay helped Cooper-pair crosses the primary Josephson junction barrater of zinc ions, and the secondary barrier of the virus and induced a high frequency oscillation. The relay turns on the superconductive state after S1 virus strongly chelated to ABS02.

### Example—8 the S1 SARS-CoV2 Virus Inhibitor as a Valve Turned the Biomimetic ACE2 Mems-Element

### Sensor 1 to a Superconductive Oscillator Sensor

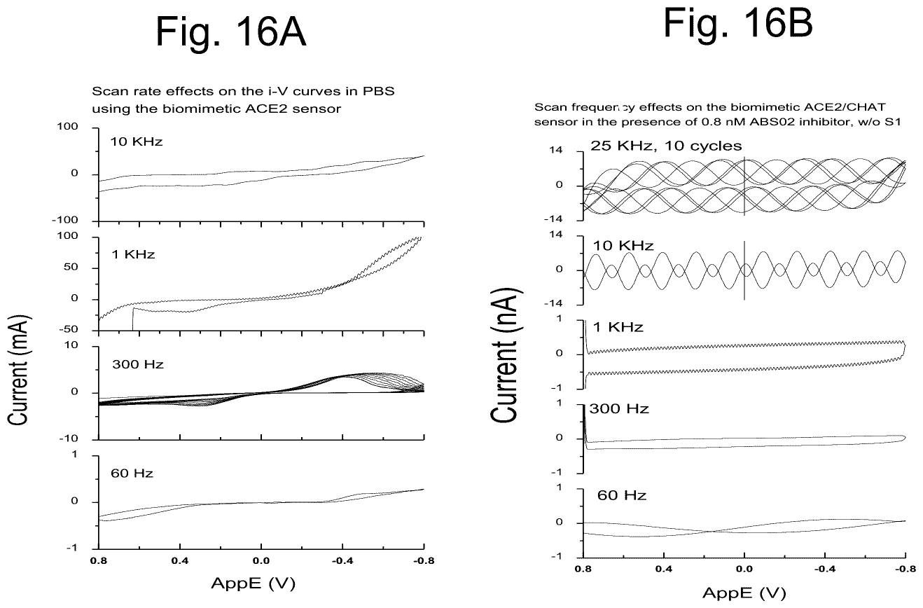

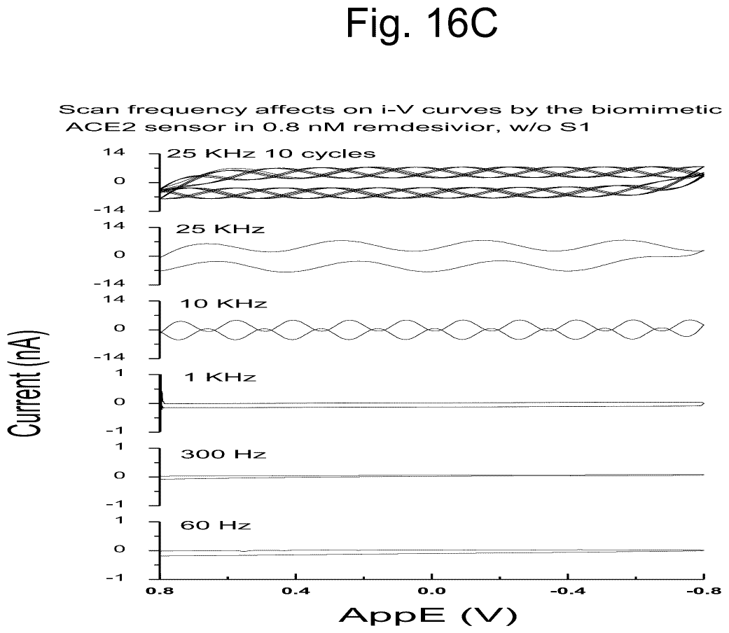

FIG. 16A depicts the scan rate impacts on the biomimetic ACE2 Sensor 1's i-V curves from 60 Hz to 10 kHz in the PBS solutions as the controls. The memristive curves are observed from 60 Hz to 1 kHz with the cross points passed through V=0, and i=o with the butterfly's loops when consecutive scans were conducted as shown in 300 Hz scan rate. This means Sensor 1 is a “healthy” normal sensor at low scan rate has the memory function [23-25]. The i-V curve at 10 kHz shows a capacitive nature without a memristive loop. FIG. 16B depicts the scan rate impacts on the biomimetic ACE2 Sensor 1's i-V curves from 60 Hz to 25 kHz in the presence of 0.8 nM S1 inhibitor ABS02 in the PBS solution. The i-V curves shown the superconductive anharmonic oscillation are observed from scan rate 10 kHz to 25 kHz with 0.8 nM ABS02 without the presence of virus, and the curves show superposition at P₁(0, δ) and P₂ (0, −δ), and we also see an 180° phase change from the forward scan to the backward scan at 10 kHz. The supercurrent intensity in FIG. 16B is 7 nA at 10 kHz with 0.8 nM ABS02 inhibitor only, without virus, compared with the signal intensity 4.0 mA in the buffer control at 10 kHz shown in FIG. 16A, indicates the superlattice 3D-cage structure with Josephson toroidal junction array and the ABS02's function and structure caused superconductive oscillation is dominative over Sensor 1's original mems-element function. The curves at 25 kHz are for 10 consecutive scans, indicate the superconductive anharmonic oscillations having superpositions. For scan rate at 1 kHz and 300 Hz, there was no superconductive nor hysteresis loop observed in FIG. 16B, but a topological insulator behaviors are observed with the envelop center located at the v=0 and i=0. At 60 Hz, the hysteresis loops i-V curve was observed. FIG. 16C depicts the scan rate impacts on the biomimetic ACE2 Sensor 1's i-V curves from 60 Hz to 25 kHz in the presence of 0.8 nM S1 inhibitor Remdesivir in the PBS solution. The superconductive oscillation curves suppressing S1 sensor's current were observed at 10 kHz, 20 kHz, and 25 kHz, but less strength compared with ABS02's effect. There was no hysteresis loop at 60 Hz, the topological resistive i-V curves indicate REM may not be able to regain memory at low scan rate. The appealing feature of blocking S1 virus by the two inhibitors is based on the high Josephson frequency superconductive oscillation and the unique membrane structure, herein the inhibitors are able to shielding the attack from S1 virus from a single virus particle to 120 nM concentration.

### Example-9 Quantitatively Evaluation of the S1 SARS-CoV2 Inhibitors Protection Ability from the S1 Virus Attack in Clinical Concentration Ranges Using Sensor 1 by the CV Method

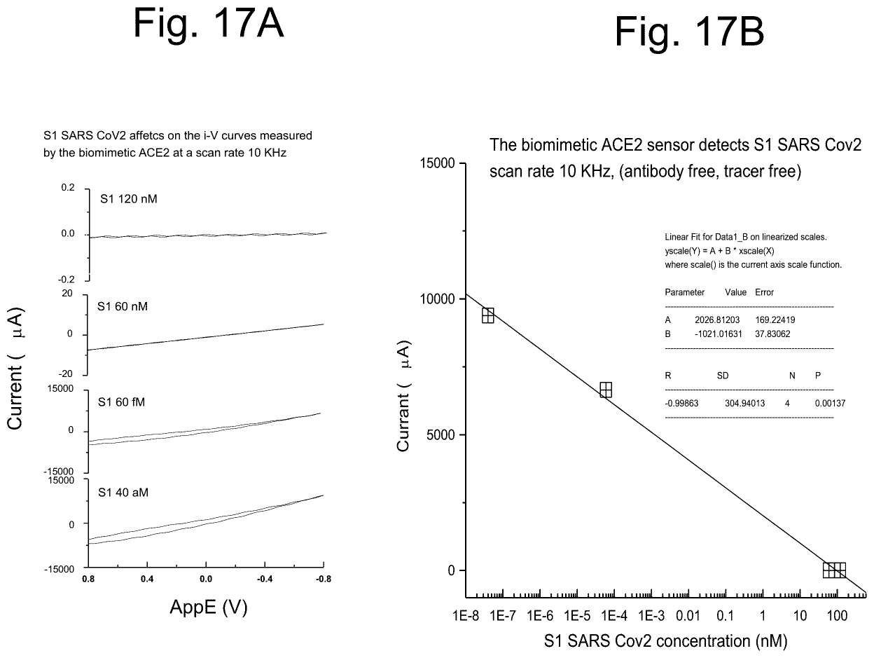

FIG. 17A depicts the S1 SARS-CoV2 virus concentration change between 40 aM to 120 nM effects the current intensity of the i-V curves at a scan rate 10 kHz compared with the i-V curves in the controls of the PBS solution using Sensor 1 shown in FIG. 16A. FIG. 17B depicts the semi log calibration curve with the current intensity inversely proportional to the S1 virus concentration by the CV method with y (μA)=2026.8 (μA)−1021 (μA/nM)·log X with r=−0.9986, SD 304.9, n=4, P<0.001. This interpreted as if 1.0 nM S1 concentration change in increases in X, that is associated with a −1021±37.83 current (in μA) change in the value of Y at 10 kHz compared with 40.54 mA of the MEMS-peak from the buffer control at X=0 without an inhibitor. In other word, if S1 concentration changes 1% increase (in X), that associated to a (−1021±37.83)/100 change in Y value.

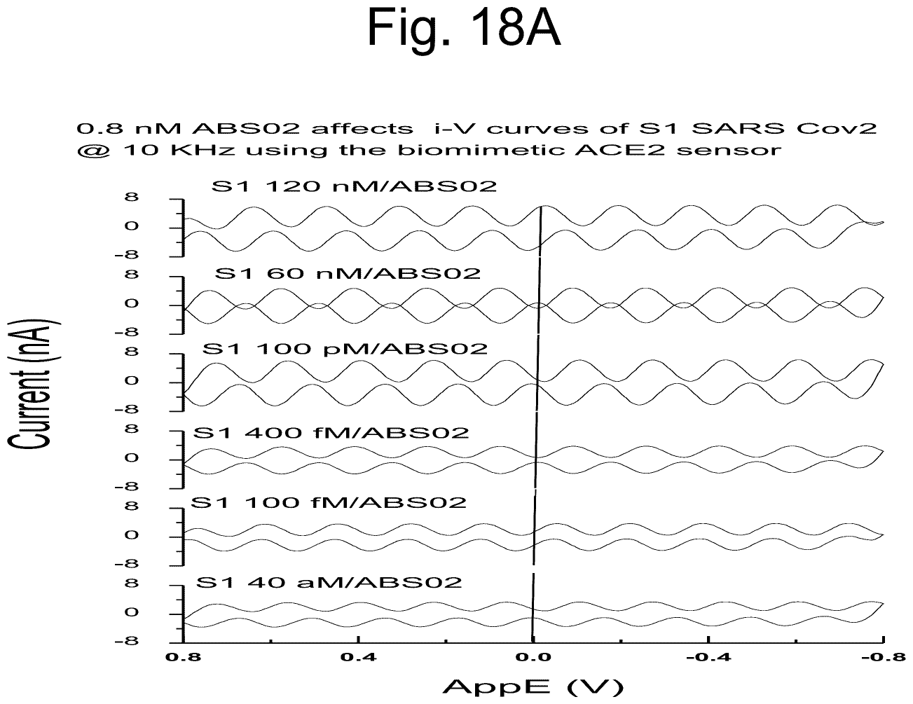

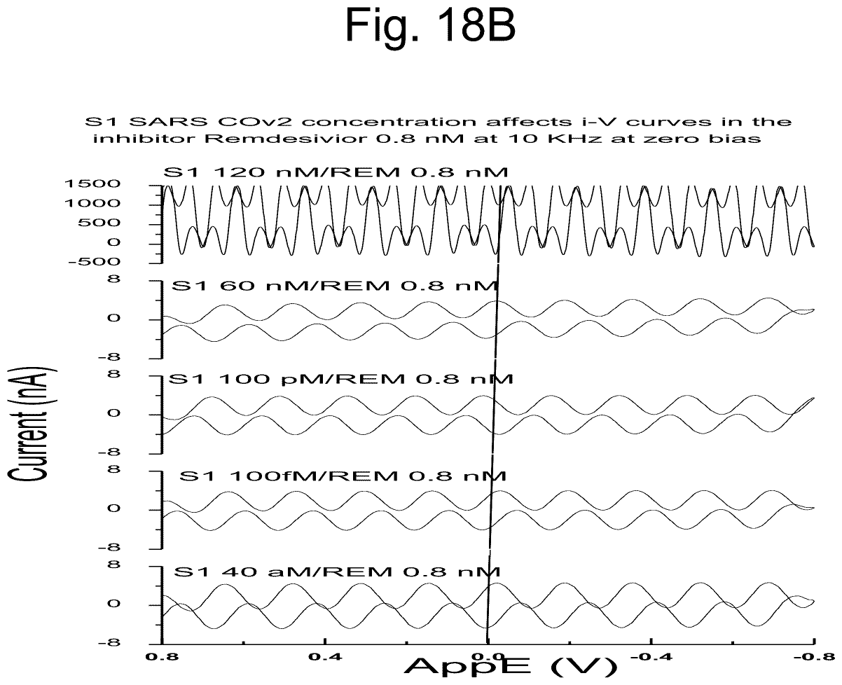

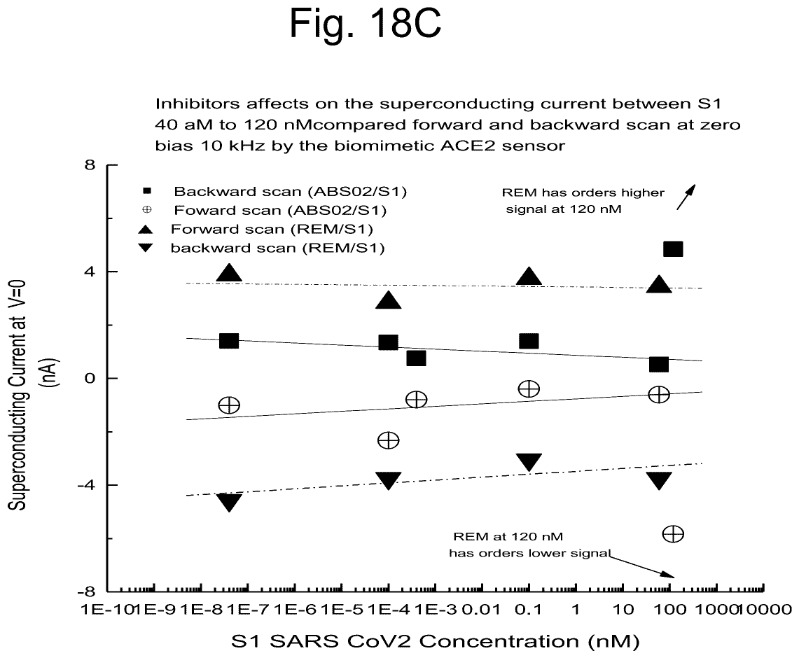

FIG. 18A depicts the superconducting oscillating i-V curves of S1 CoV2 virus over concentration range 40 aM-120 nM in the presence of the inhibitor ABS02 with a concentration 0.8 nM at a scan rate 10 kHz using Sensor 1, the supercurrent intensity at v=0 reduced as low as 10⁷ mA compared with the control, indicates the inhibitor suppressed the S1 virus communication with Sensor 1 membrane, and protected ACE2 by increase the quantum potential energy due to the Josephson phase change rate increases. FIG. 18B depicts the i-V curves of S1 CoV2 virus over concentration range 40 aM-120 nM in the presence of the inhibitor Remdesivir with a concentration 0.8 nM at scan rate 10 kHz. The similar trend was observed as in FIG. 18B. FIG. 18C depicts the semi log plots of superconducting current at V=0 vs. S1 SARS-CoV2 concentrations over 40 aM-120 nM in the presence of 0.8 nM ABS02, or 0.8 nM Remdesivir, respectively compared with the control calibration curve shown in FIG. 17B. With 0.8 nM ABS02 and 0.8 nM REM blocked S1 virus 100% over the studied range evidenced by an elimination of the S1 virus's MEMs peak completely, changed to a supercurrent oscillation sine peak at zero-bias bearing a non-linear Josephson magnetic energy due to the presence of RF SQUID loop junction induced Josephson inductance L, This non-linear magnetic energy could be a great tool to eliminate the S1 virus toxins. This means by using Sensor 1, the inhibitors blocked 100% S1 virus as low as a single virus particle, and as high as 120 nM concentration.

### Example-10 Quantitatively Evaluation of the S1 SARS-CoV2 Inhibitors Protection Ability from the S1 Virus Attack in Clinical Concentration Ranges Using Native ACE2 Sensor 2 by the CV Method

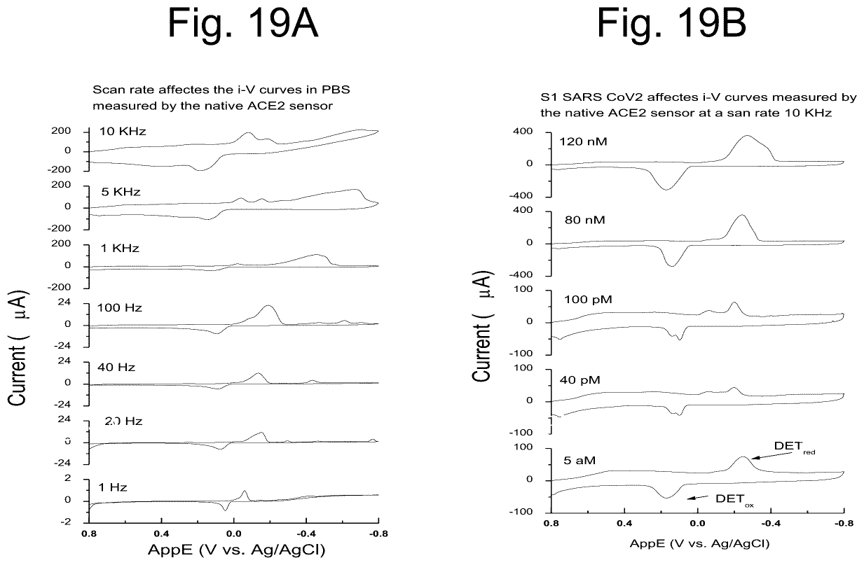

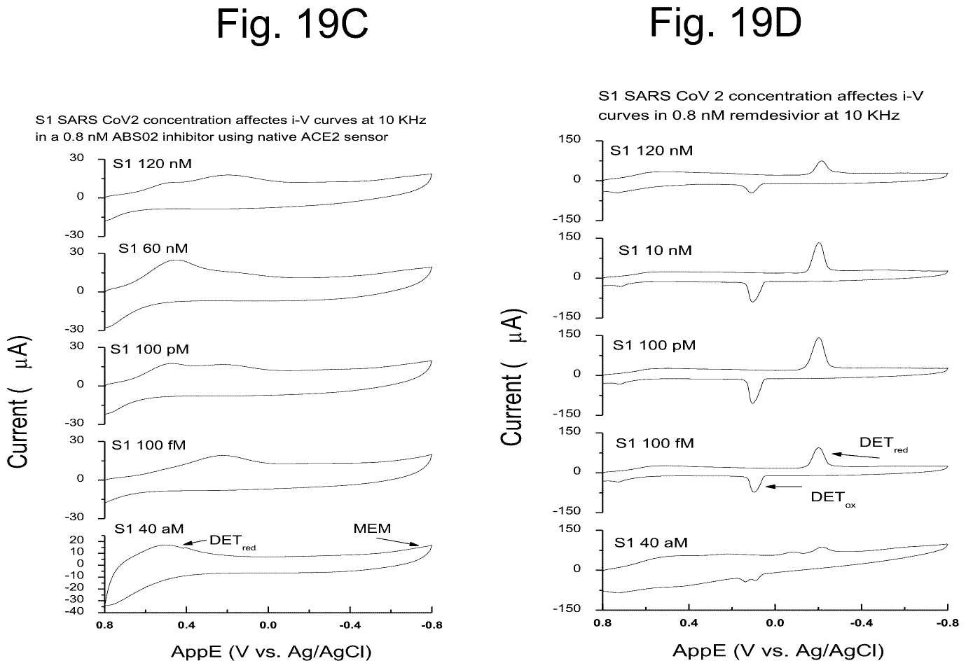

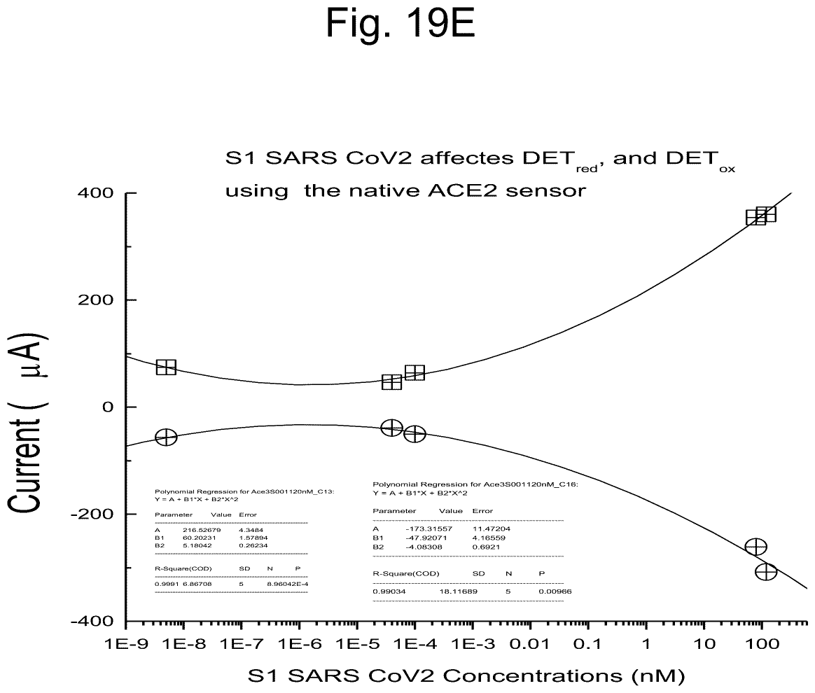

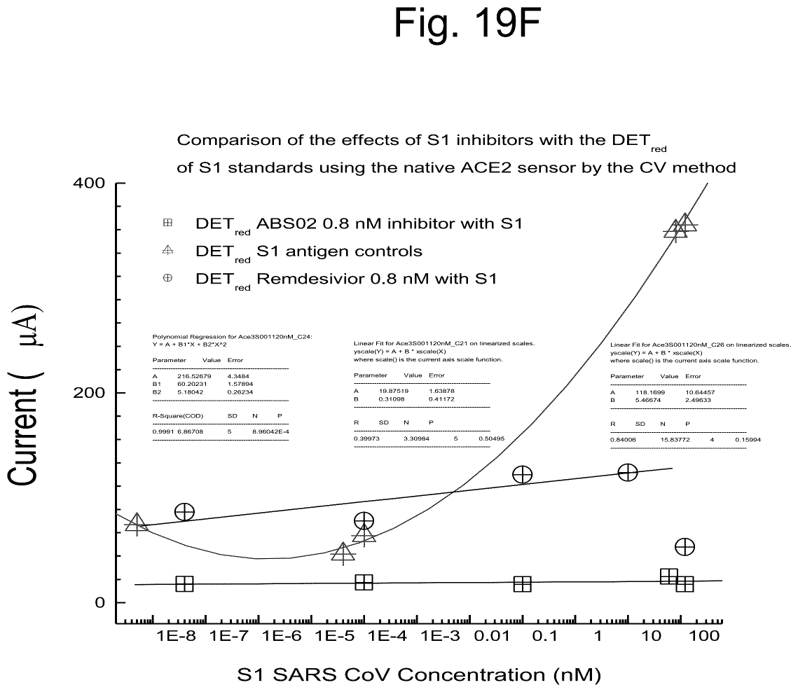

We used 0.8 nM ABS02 and 0.8 nM REM accessed the effectiveness of the inhibitors in the presences of S1 virus over 40 aM-120 nM. FIG. 19A depicts i-V curves of Sensor 2's controls in PBS solutions from scan rate 1 Hz to 10 kHz, the typical memristive curves with crossing-loop passed zero bias at v=0, and zero current at i=0, indicates Sensor 2 of the native ACE2 sensor is also a “healthy” memristive sensor. The charge on the Direct Electron Transfer (DET_{red}) peaks is proportionally increased as the scan rate increases, which is stronger than the increase of the DETₒₓ as shown in FIG. 19A. It is also noticed that the peak potential distance between the DETₒₓ and DET_{red} increased as the scan rate increases. FIG. 19B shows the trend of S1 SARS-CoV2 virus effects on the i-V curves over 5 aM-120 nM at scan rate 10 kHz compared with the control in FIG. 19A at 10 kHz, the S1 virus has exponentially increased the peak current intensity as the S1 concentration increased was observed. S1 virus significantly broken the Sensor 2's native memristive state.

The S1 virus inhibitors effect on the i-V curves of Sensor 2 presented in FIG. 19C and FIG. 19D for 0.8 nM ABS02, and 0.8 nM REM in the presence S1 virus 40 aM-120 nM at 10 kHz, respectively. In FIG. 19C there was no DETₒₓ peak observed, means the ABS02 eliminated the S1 virus critical biocommunication with native ACE2, the DET_{red} peak is moved far from the original potential spatial position. FIG. 19D shows the DETₒₓ and DET_{red} peak current intensity decreased by several fold compared with control in FIG. 19B at 10 kHz over 40 aM-120 nM. This indicates 0.8 nM REM inhibitor worked well to block the S1 communication.

There are two calibration curves for S1 SARS-CoV2 virus presented in FIG. 19E over S1 concentration 4 aM-120 nM with fittings of polynomial regression using native ACE2 Sensor 2. Sensor 1's sensitivity to S1 virus interaction is 16.96-fold higher than Sensor 2. FIG. 19F compares 0.8 nM ABS02 and 0.8 nM REM S1 inhibitors blocked S1 virus DET_{red} peaks over 40 aM to 120 nM by 99.5±0.4% as the symbols fittings with semi log, and 90.9±2.5% as the ⊕ symbols with a semi log fitting result compared with the sensitivity from the controls as the Δ symbols with red line of polynomial fitting result shown in the FIG. 19F at 10 kHz, respectively. The results show ABS02 has a super performance compared with REM in fighting from S1 SARS-CoV2 virus through wide concentration range.

### Example-11 Validation S1 Inhibitors' Performance Using an Open Circuit Potential (OPO) Method by the Biomimetic Sensor 1

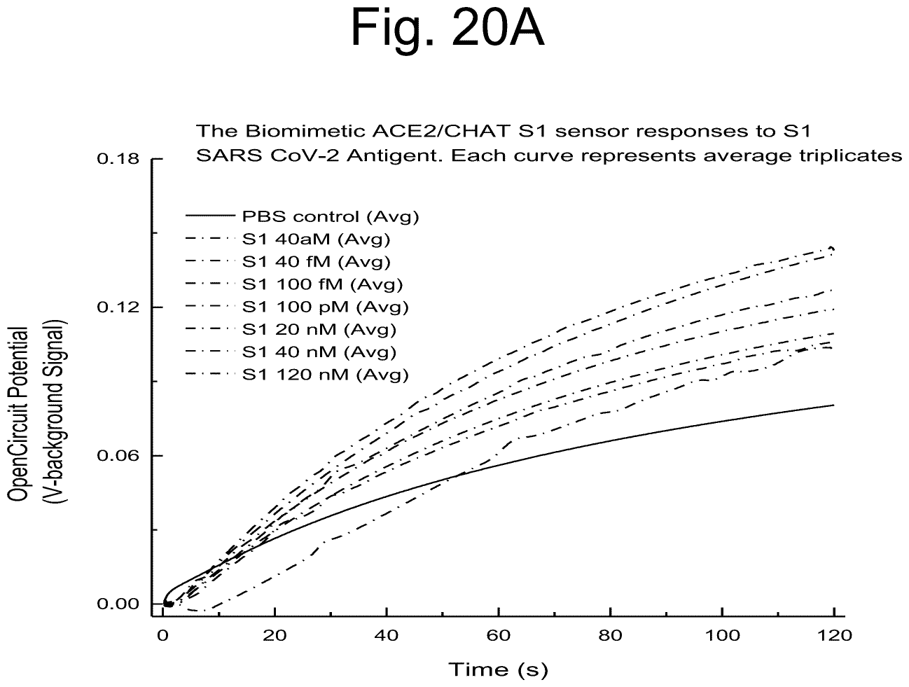

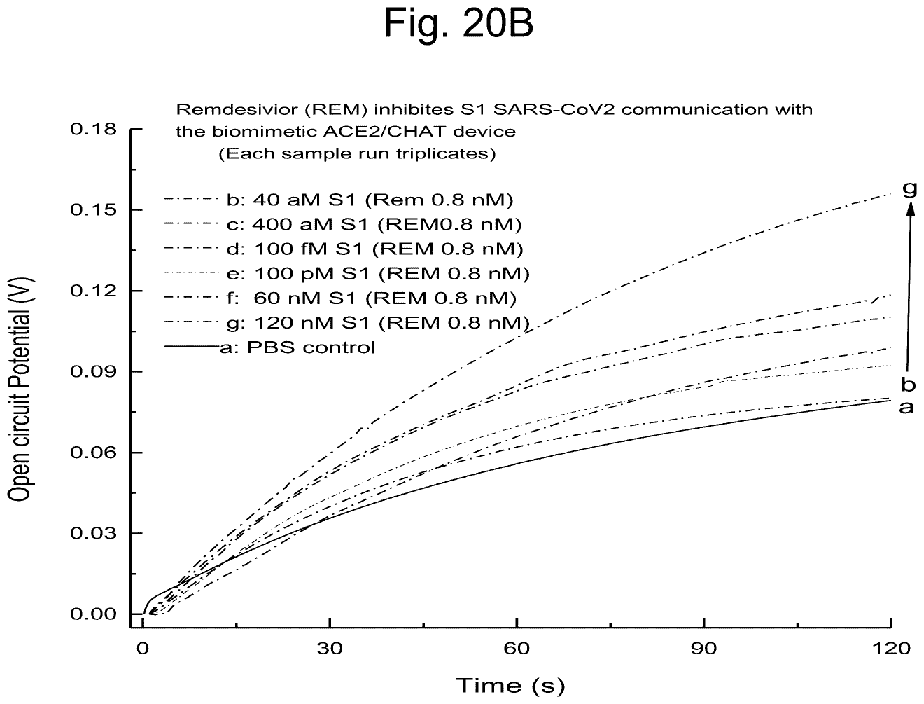

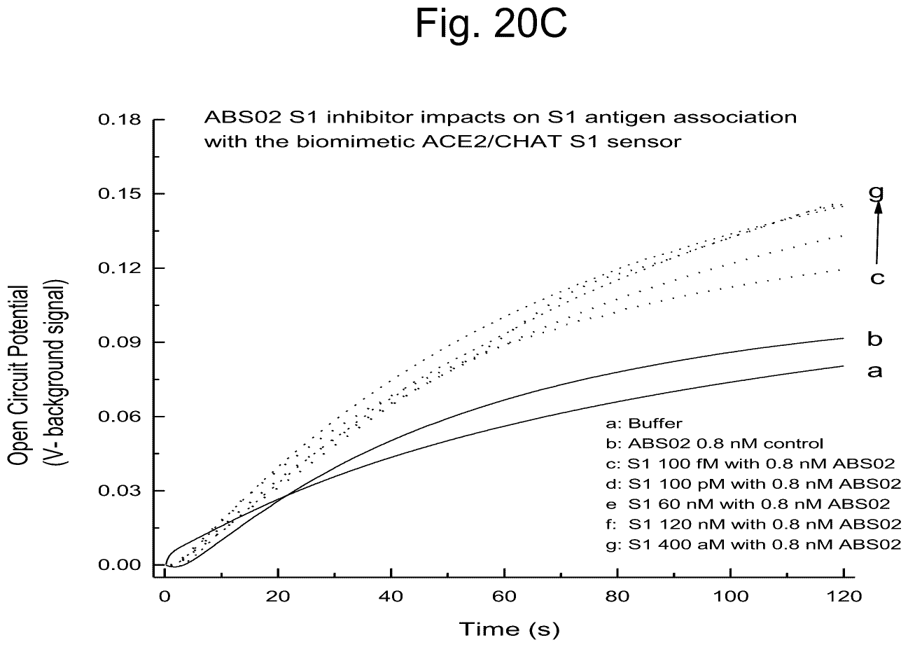

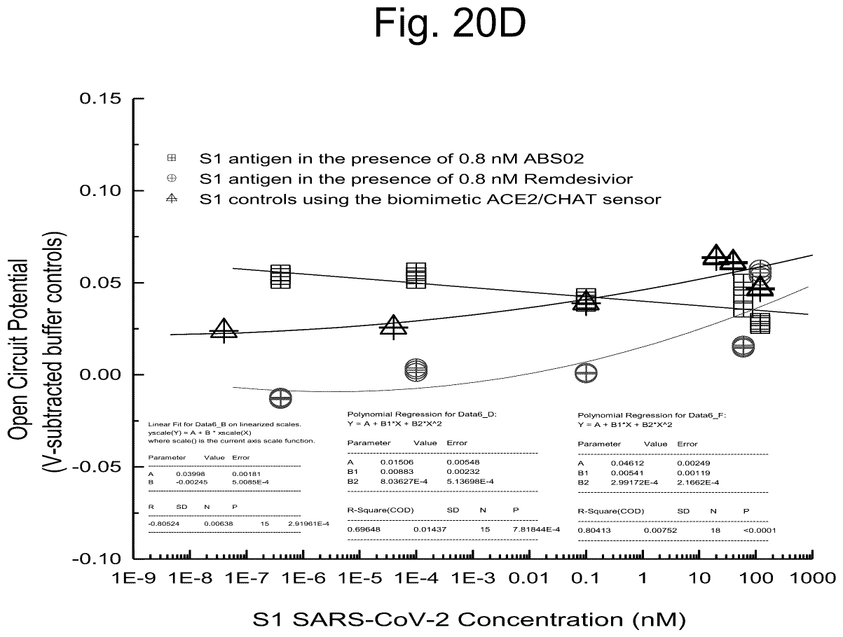

The OPO method has been used for accessing a system's equilibrium energy with open circuit potential testing, just like people used to test a battery's voltage. Our group had research articles and patents published using one of the OPO method [26-30]. FIG. 20A depicts the plots of the signal of open circuit potential curves vs. time after subtracted the background for S1 SARS-COV2 concentration over 40 aM-120 nM over 7 concentration levels against the PBS controls, with each sample run triplicates using the biomimetic ACE2 Sensor 1 before applying an inhibitor. FIG. 20B depicts the plots of the signal of open circuit potential vs. time using the biomimetic ACE2 Sensor 1 after applying a 0.8 nM inhibitor Remdesivir samples under the same experimental conditions as in FIG. 20A. FIG. 20C depicts the plots of the signal of open circuit potential curves vs. time in the presence of 0.8 nM ABS02 inhibitor compared with the controls under S1 concentration 400 aM-120 nM using the biomimetic ACE2 Sensor 1. FIG. 20D depicts the semi log calibration curves of the OPO potential of the S1 SARS-CoV2 virus vs. time using Sensor 1 over 40 aM-120 nM after 0.8 nM ABS02 inhibitor ( [symbol] ), and 0.8 nM Remdesivir (⊕) applied, respectively compared with the S1 control curve (▴). Results of the models of fittings were presented in FIG. 20D, the results of the S1 SARS-CoV2 virus' sensitivity are reduced by 55% by 0.8 nM ABS02 in the presence of S1 over studied range compared with the S1 control samples' sensitivity without inhibitor, and the 0.8 nM REM increased the sensitivity by 63% compared with control sensitivity in the presence S1 virus concentrations, indicates reducing the capacitive energy caused by S1 virus' inflammation to avoiding damage the healthy cell is an appropriate approach, because Sensor 1 in the control PBS solution has the lowest OPO potential as shown the control curves in FIGS. 20A, 20B and 20C, respectively.

### Example-12 Validation S1 Inhibitors' Performance Using an Open Circuit Potential (OPO) Method by the Native ACE2 Sensor 2

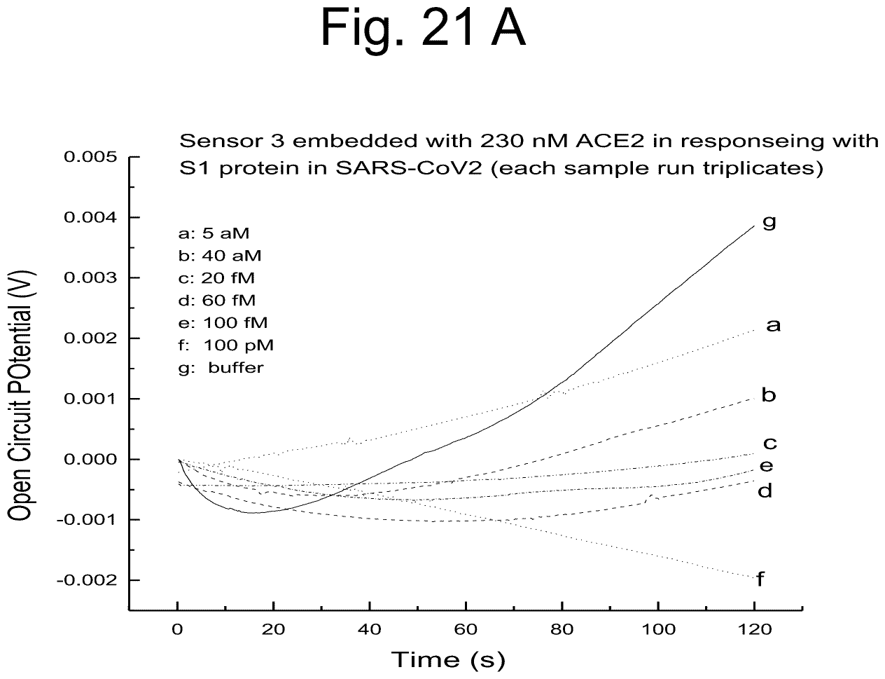

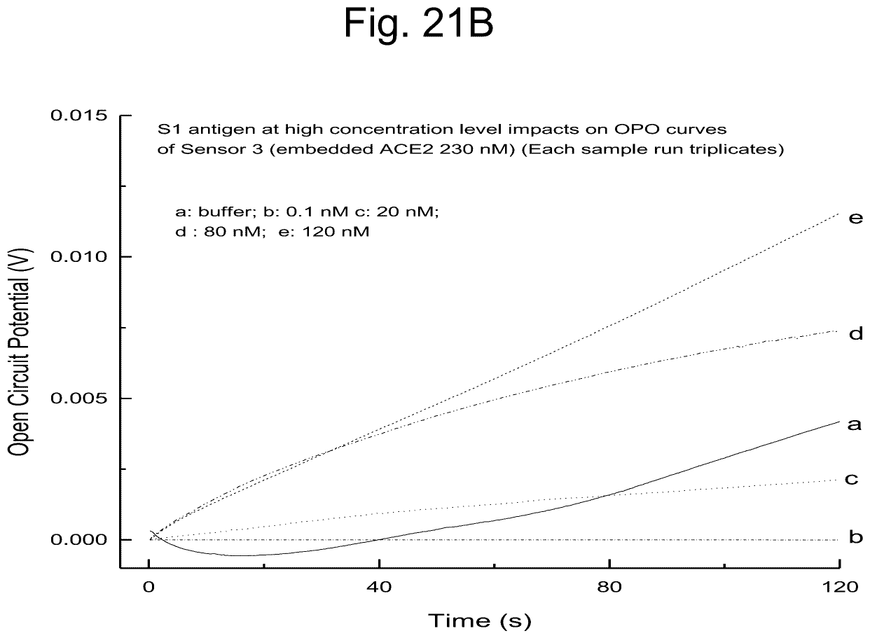

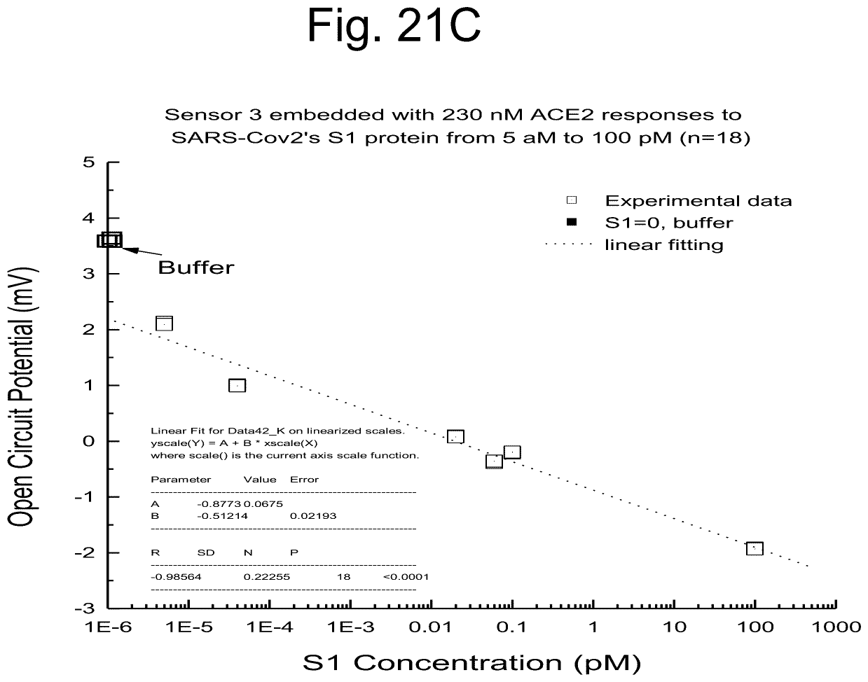

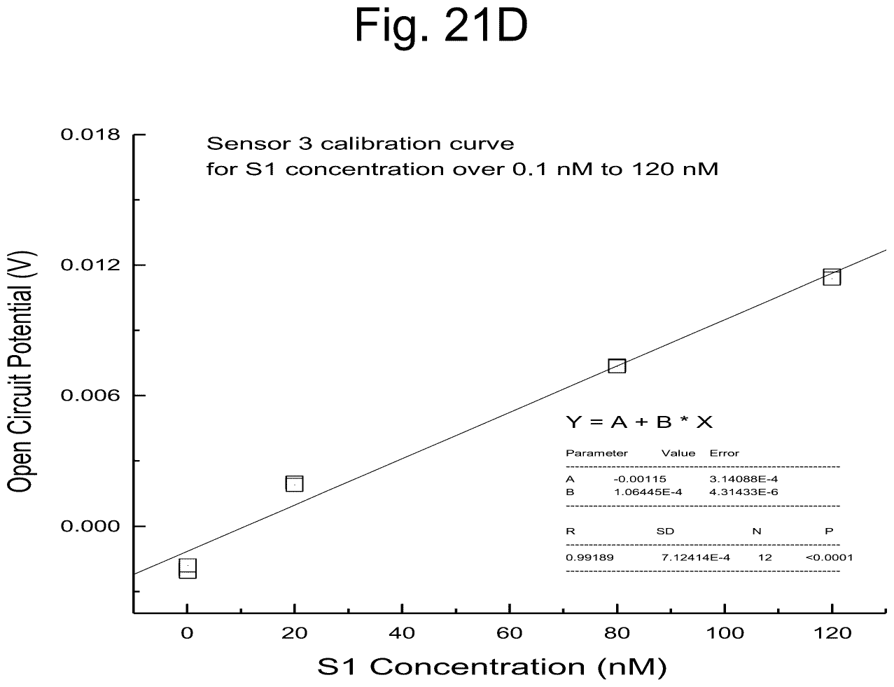

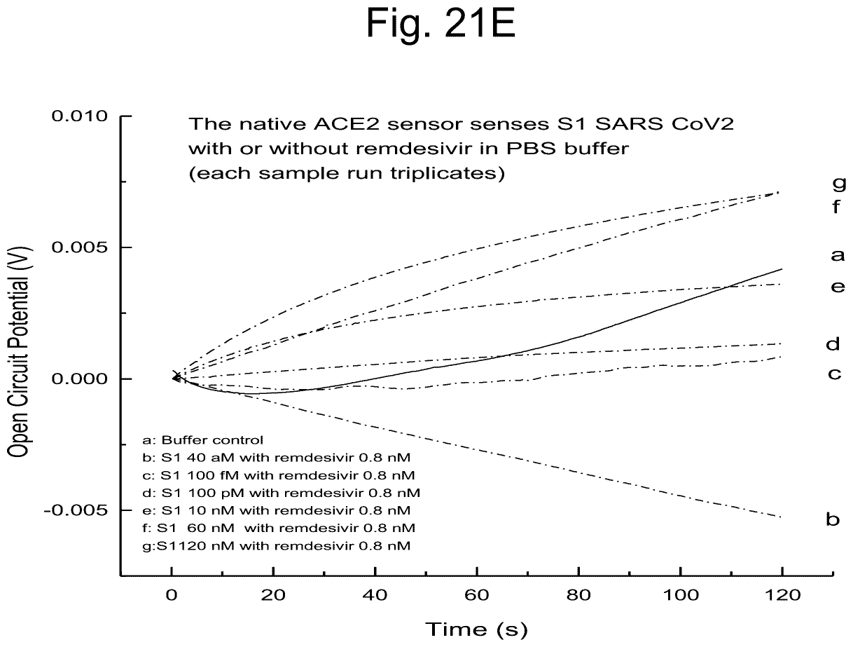

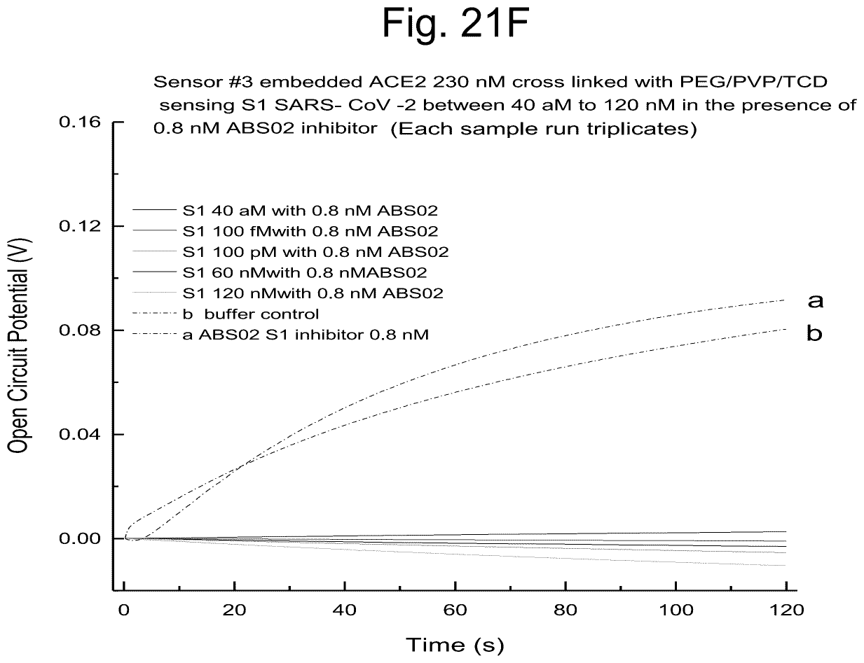

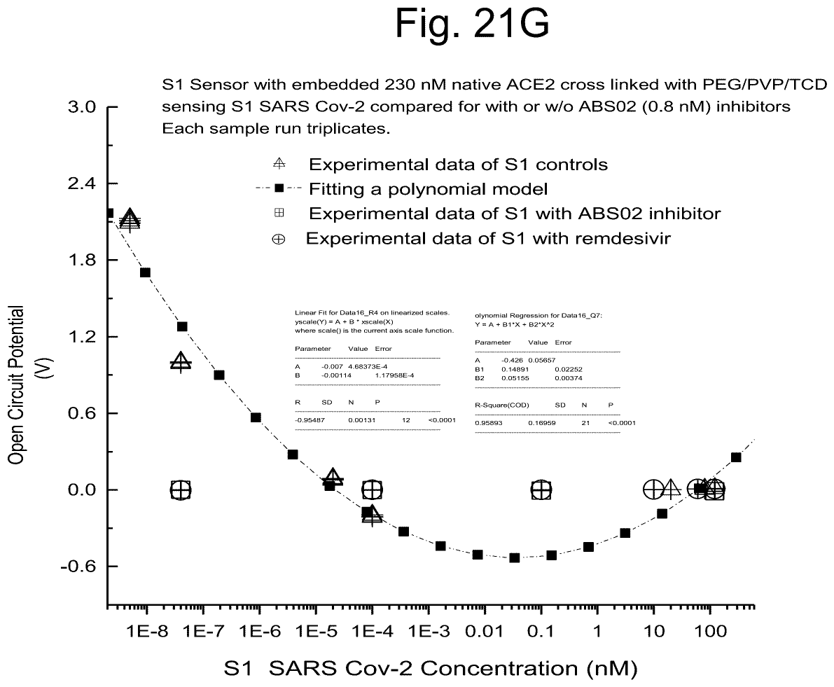

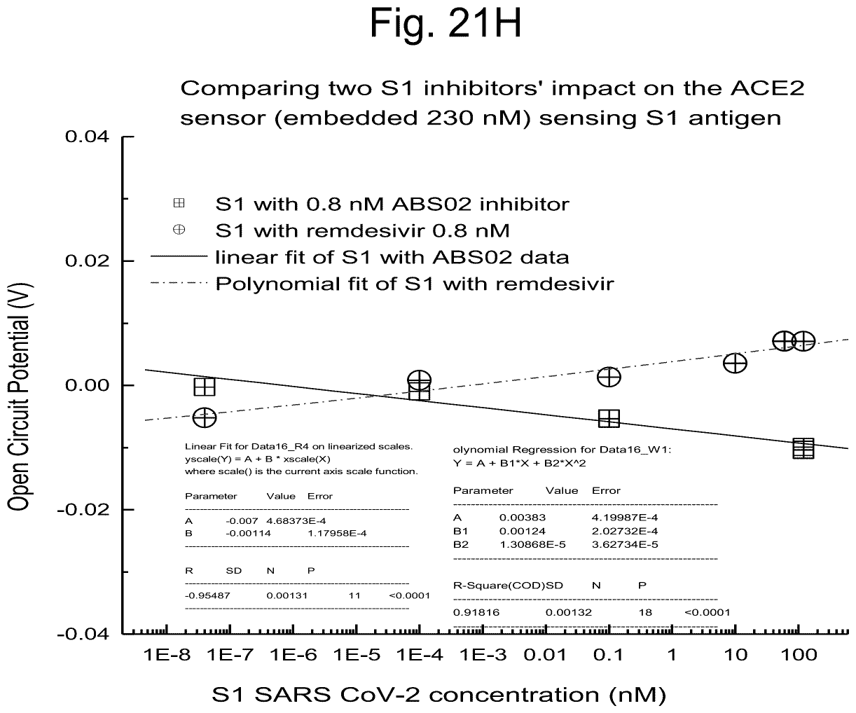

FIG. 21A shows in the presence of S1 virus, the OPO potential was inversely proportional to the S1 concentration from 5 aM to 100 pM of 6 levels compared with controls, and the negative energy started appear at 100 fM. FIG. 21B depicts S1 concentrations inversely impact Sensor 2's voltage at the higher end over 0.1 nM to 120 nM of 4 levels.

FIG. 21C depicts the calibration curve at the low end of S1 concentration vs. OPO potential compared with the controls using the native ACE2 Sensor 2. It produced a log regression equation Y (mV)=−0.877−0.51 Log X, r=−0.986 (n=18), P<0.0001, Sy/x=0.22 over S1 concentration 5.0 aM to 100 pM having an imprecision of the single virus particle of 0.1%. The Detection of Limits (DOL) is 0.19 aM. FIG. 21D depicts the calibration curve at the high end. It produced a linear regression equation Y (mV)=−1.15+0.1 X, r=−0.992 (n=12), P<0.0001, Sy/x=0.71 over S1 concentration 0.1 nM to 120 nM with a DOL 0.5 nM having an imprecision of 1.96%.

The S1 virus inhibitors effect on the voltage vs. time curves in the presence of S1 SARS-CoV2 concentrations over 40 aM-120 nM compared with the buffer controls presented in FIG. 21E with 0.8 nM REM inhibitor and FIG. 21F with 0.8 nM ABS02 inhibitor in the Se virus over 40 aM-120 nM compared with the controls, respectively. It was noticed that both inhibitors have considerably reduced S1 virus voltage, especially 0.8 nM ABS02 has eliminated almost all S1 signals over the entire S1 virus study range compared with control signals using the native ACE2 Sensor 2 in FIG. 21F, by contrast, 0.8 nM Rem inhibitor did not reduce as much as the ABS02 reduced.

FIG. 21G depicts the inhibitors at 0.8 nM concentration effect on the voltage through the plots compared with the S1 virus standard curve, indicate ABS02 and REM are 99.23% and 99.16% blocked S1 activity compared with controls, the results of model fitting attached in FIG. 21G over 40 aM-120 nM for S1 concentration with triplicates run for each sample. It is appeared voltage curves vs. S1 concentrations are flat overlapped for the both inhibitors with voltage values oriented at zero voltage over the entire S1 range, but the S1 standard curve is a curvature with voltage up to 2.1V and down to −0.6V over the studied S1 virus range. This indicates the two inhibitors are worked effectively against S1 virus over a wide range, and effectively protected the cell. FIG. 21H shows the detail plots of the open circuit potential vs. S1 virus concentration over 40 aM-120 nM in the presence of 0.8 nM ABS02 and 0.8 nM REM, respectfully. After conducting a two-tailed statistical analysis of the slope with H₀:β₁=0, the result calculated has not rejected the hypothesis from the case of ABS02 inhibitor, herein S1 produced no signal different from the regression through origin over the wide range, which is advantages from REM inhibitor, failed the statistic test of regression through origin.

### Example-13 Comparing the Performance of the Two S1 Inhibitors in the Power of Restoration of Reversible Membrane Potential (RMP) by the Double-Step Chronopotentiometry (DSCPO) Voltage Method Using Sensor 1

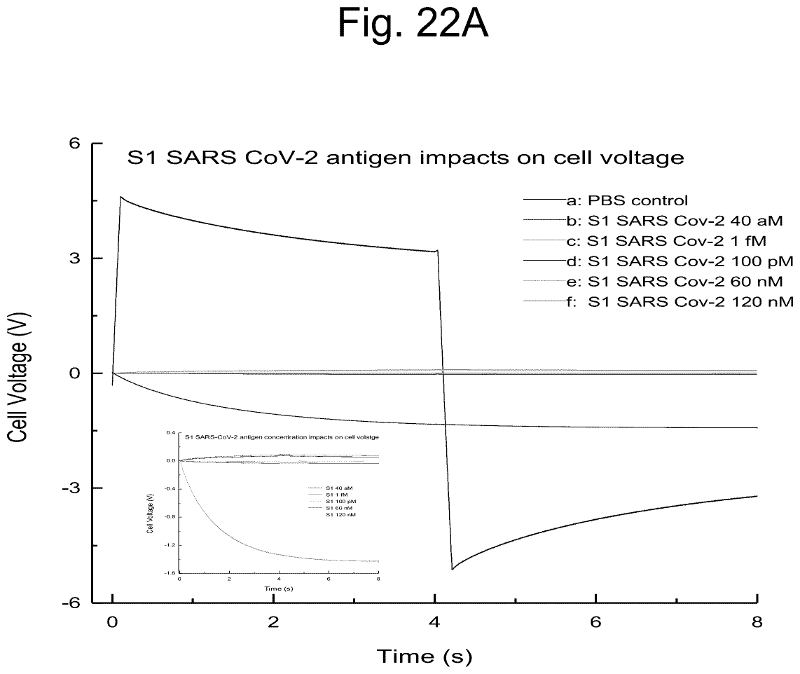

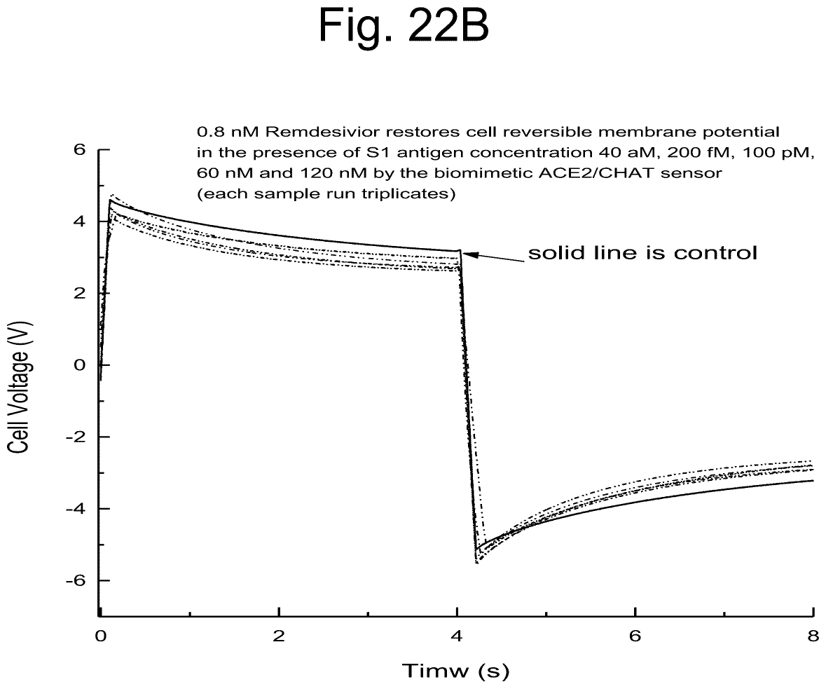

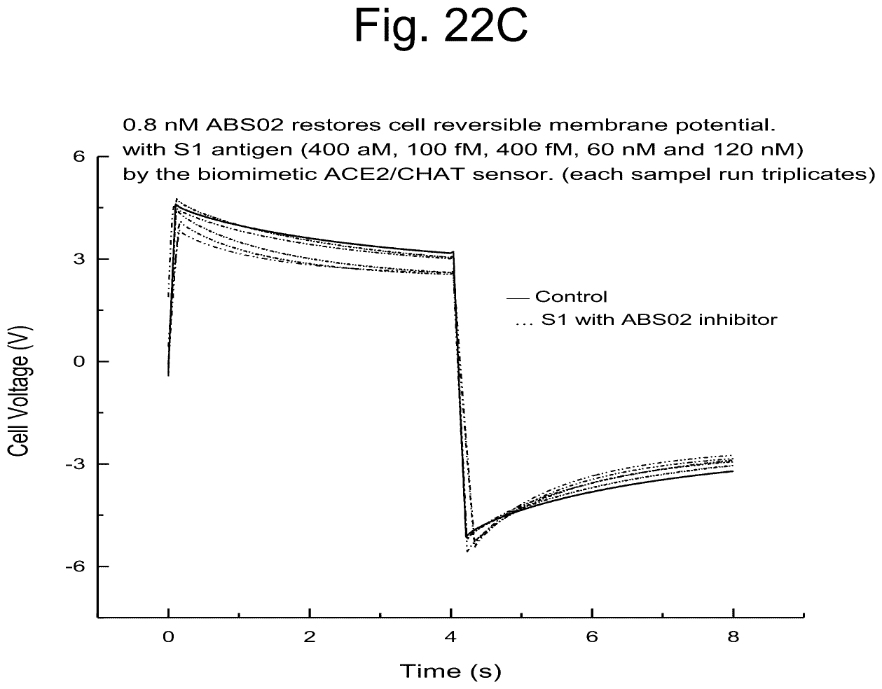

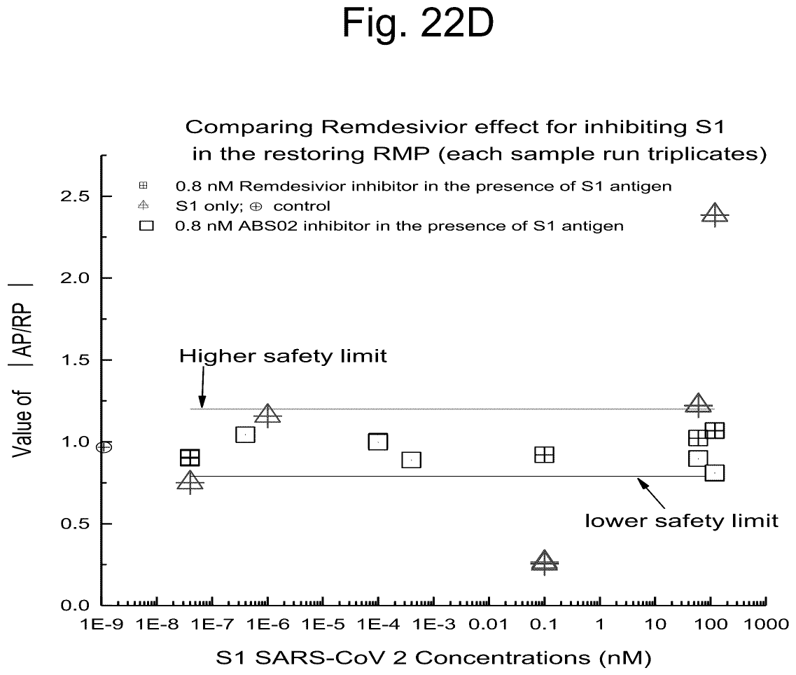

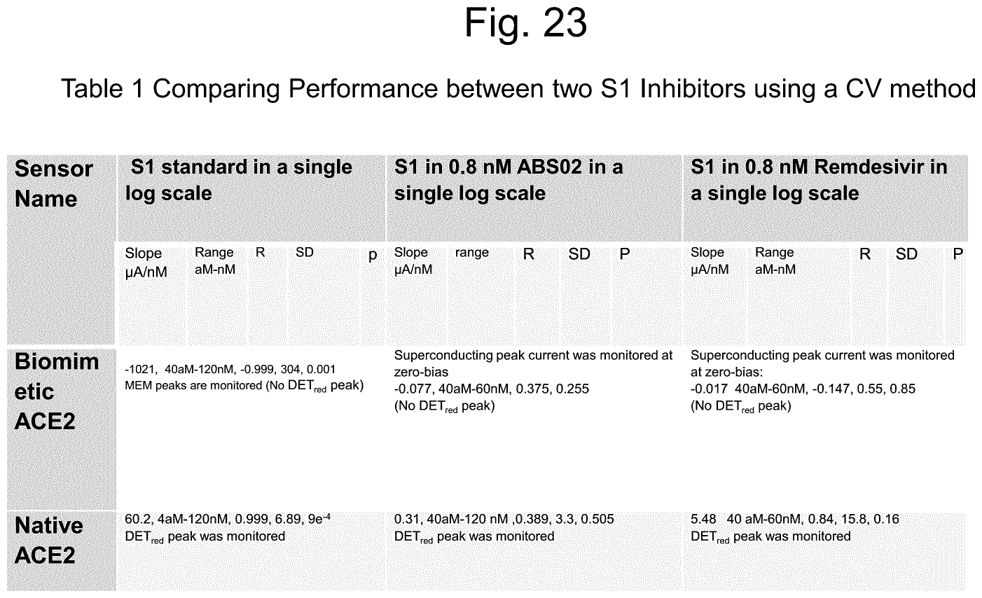

Researchers reported many diseases unable to maintain mitochondrial cell's RMP, and a biomarker of the potential ratio of Action potential (Ap) vs. resting potential (Rp) found is an indicator that direct correlating with the RPM [31-36]. FIG. 22A depicts sensor 1 has the Ap/Rp ratio results located in the unsafe zone due to S1 virus without the applying an inhibitor compared with the puffer controls over S1 concentration 40 aM-120 nM with five levels by using the DSCPO method. The insert is the enlarged curves at lower S1 concentration. Each sample run triplicates. FIG. 22B depicts the voltage vs. time curves in the presence of 0.8 nM Remdesivir over the same S1 concentration range between 40 aM-120 nM compared with controls. The curves show REM inhibitor has blocked S1 virus and maintained the energy compared with controls over a wide virus range. FIG. 22C depicts the voltage vs. time curves in the presence of 0.8 nM ABS02 over the same S1 concentration range compared with controls. The similar voltage vs. time curves was observed after applied ABS02 inhibitor as the REM did to maintain a healthy energy profiles over a wide virus range.

FIG. 22D depicts the results for the restoration of the RMP used the biomimetic ACE2 sensor 1 by the voltage method, 0.8 nM ABS02 and 0.8 nM Remdesivir both (n=15 for each inhibitor) have ratio values 100% located in the safety zoon over S1 40 aM to 120 nM, but S1 SARS-CoV2 (n=18) alone has more than 50% data located outside of the safety zoon.

Multiple groups suggested to use salivary samples for testing SARS-CoV-2, because of the easiness for self-collected sample with almost no discomfort [37-38]. literature reported the human specimen accuracy study for S1 SARS-CoV2 revealed human fasting saliva samples with or without spiked 40 aM S1 antigen showed the recovery rate (accuracy) is 96±4% [15]. The recovery results showed 99±2% using the NIST SRM965 human serum with a certified glucose 300 mg/dL as the control, compared with spiked S1 antigen 60 nM in the serum by the OPO method [15].

The fasting salivary samples were collected from a healthy subject, and went through the Board approval. The point imprecision and accuracy were assessed by the recovery study. The results showed the recovery rate is 92.3±9% by spiked S1 antigen 40 aM samples against the saliva controls after corrected the factor between the saliva controls and the buffer controls. The recovery results showed 118±0.2% using the NIST SRM965 human serum with a certified glucose 300 mg/dL as the control, compared with spiked S1 antigen 60 nM in the serum by the DSCPO method [15].

### Example—14 Summary of the Results Comparing the Effects from the Inhibitors Blocking the S1 SARS-COV2 by Regression Analysis Based on the Data Obtained Using the OPO Method

Results of comparison two inhibitors' performance using an OPO method based on Sensor 1 and Sensor 2 for with or without the presence of inhibitors against the same studied S1 concentration ranges indicate: (1) case 1 for without the inhibitors, Sensor 1's sensitivity for detection of the S1 virus reduced the eternal capacitive energy by 96.4% compared with the sensitivity of the native ACE2 Sensor 2 detection of S1 over concentration range 40 aM-120 nM (n=18) and 4 aM-120 nM (n=21), respectively. It indicates the biomimetic ACE2 Sensor 1 in the presence of a wide range S1 Cov19 virus prevented the S1 spike SARS-CoV2 virus gaining energy from the environment (from the patients' cell energy). Just had a similar event happened reported in the literature that using the OPO method detected the presence of bacteria through monitoring the curves of the open circuit potential at different ATP concentrations vs. time, and built a calibration curve over 25 aM to 400 pM ATP, the results show an exponential increase profile in a semi log plot using a superconductive biomimetic protein sensor under the antibody-free and reagent-free conditions [38-39]. The present invention makes a progress with 97.2% reduced the S1 virus' sensitivity to the intrinsic energy compared with the sensitivity of ATP caused OPO energy increase rate [39]. Sensor 1 for without an inhibitor, has 96.4% reduced the sensitivity of S1 virus energy compared with Sensor 2, the native ACE2 sensor 2 over 40 aM-120 nM (n=18), and 4 aM-120 nM (n=21), respectively. (2) the Case 2, with the inhibitors, under the impact of 0.8 nM REM inhibitor, the intrinsic energy gain rate increased by 63% detected by Sensor 1 over 400 aM-120 nM (n=15), but reduced by 99.17% detected by Sensor 2 (n=18) over 40 aM-120 nM. Under the impact of 0.8 nM ABS02 inhibitor, the S1 virus intrinsic energy gaining rate reduced by 100% detected by Sensor 1(n=15) over 400 aM-120 nM, and it reduced the S1 virus energy gaining rate by 100% detected by Sensor 2 (n=12) over 40 aM-120 nM.

### Example-15 Summary of the Results Comparing the Effects from the Inhibitors Blocking the S1 SARS-COV2 by Regression Analysis Based on the Data Obtained Using the CV Method

FIG. 23 Table 1 summarizes results for comparison of the regression results between two S1 inhibitors effeteness for blocking S1 SARS-CoV2 virus biocommunication with the two sensors based on the data obtained using the CV method. For the case 1 without the inhibitors, Sensor 1's sensitivity to detect the S1 antigen over 40 aM-120 nM is 16.96-fold sensitive than Sensor 2. (2) For the Case 2, under the impact of 0.8 nM REM inhibitor, Sensor 1 blocked S1's biocommunication 100%, compared to the native ACE2 sensor 2, which reduced by 90.9% when the 0.8 nM REM presented in the same S1 range 40 aM-120 nM. The inhibitor ABS02 blocked S1 signal by 100% related to the original sensitivity without an inhibitor. It appears both inhibitors blocked S1 virus's attack 100% for Sensor 1, but for the native ACE2 Sensor 2, is 10% short from 100% using REM. The fact revealed a truth, that is the power of the superconductivity state gained for Sensor 1 as soon as the inhibitor interacted with function groups in the cavity of the Josephson toroidal junction array, the mems-element state was switched to a superconductive quantum oscillator state at the high frequency; but Sensor 2 does not have the superlattice toroidal structure, unable to transfer from a MEMS-element state to a superconducting quantum oscillating state.

### Example—16 Conclusion

In the fight against the COVID-19 pandemic, the need for a quick and efficient screening process for potential pharmaceutical candidates is more important than ever. The device described in this invention comprises a nanostructured double-layered membrane with a biomimetic human angiotensin-converting enzyme 2 (ACE2) function. The membrane comprises a superconductive Josephson toroidal junction array (JTJA) with zinc ions serving as the primary Josephson junction insulator. This device can accurately detect the presence of a single S1 SARS-CoV2 virus protein from 40 aM concentration of up to 120 nM without the need for antibodies or labeling. Additionally, the invention identified a secondary JTJA insulator. The presence of a single flux quantum observed due to Cooper-pair long tunneling high-frequency oscillation with an S-I₁-S·I₂ (virus)·S (inhibitor) configuration, that effectively blocked virus communication and transitioning the original memristive state to a superconductive quantum state with an external magnetic field-free. The invention also allows for the identification and analysis of an appropriate virus inhibitor compared to an established Remdesivir. The identified S1 inhibitors were found to restore the function of a cell's reversible membrane potential (RMP) with 100% efficacy within a safety zone, compared to only 50% efficacy outside of the safety zone without the inhibitor. In conclusion, The biomimetic ACE2 sensor model technology invented for identify virus inhibitors and for detecting the asymptomatic virus infection is a game-changing platform technology that can revolutionize the way we detect and fight the COVID-19 pandemic. With its strong and reliable performance, we believe the platform model approaches can be a valuable tool in the global fight against this deadly virus. The potential applications are expected to identify inhibitors of virus infections on crops, fishes and animals who have an ACE2-like zinc-finger molecular structure in their cells.

### REFERENCE

### References

1. X. Liu, X. Li, D-L Deng, X-J Liu, and S. Das Sarma, arXiv, 1602.08093v2, 2016.
2. J. Li, T. Neupert, B. A. Bernevig, A. Yazdani, Nature Communications, 7:10395, 2016.
3. www.en.wikipedia.org/Josephson vortex
4. E. T. Chen, J. T. Thornton, P. T. Kissinger, and S-H. Duh, Biotech, Biomaterials and Biomedical: TechConnect Briefs 2018, 107-110.
5. E. T. Chen, J. T. Thornton, P. T. Kissinger, and S-H. Duh, TechConnect Briefs in Informatics, electronics and Microsystems 2018, 43-46.
6. E. T. Chen, J. T. Thornton, P. T. Kissinger, S-H. Duh, Sensors and Transducers, 2018, 228(12), 48-62.
7. E. T. Chen, Chapter Three, book of Nanoengineering, Quantum Science, and, Nanotechnology Handbook (Editor S. E. Lyshevski), CRC Press, ISBN Number: 978-0-367-19751-3 (Hardback), Dec. 2, 2019.
8. E. T. Chen, U.S. patent Ser. No. 11/079,354, Aug. 3, 2021.
9. E. T. Chen, J. T. Thornton, P. T. Kissinger and S-H. Duh, Sensors and Transducers 2018, 228(12), 30-47.
10. S. A. Hosseni, F. Zahedipour, H. Mirzaei, and R. K. Oskuee, Potential SARS-CoV-2 vaccines: Concept, progress, and challenges, Int Immunopharmacol. 2021 August; 97: 107622.
11. E. T. Chen, J. T. Thornton and S-H. Duh, Increase ACE2 Concentrations Embedded in the Memristive Electrochemical Sensor Membranes Promote Direct Reagent-free Sensing of S1 Antigen of SARS-Cov2 Virus, TechConnect Briefs in Biomaterials, Diagnostic and Bioimages, 111-114, 2022.
12. Afaghi, P, Lapolla, M. A., Ghandi, K., Denaturation of the SARS-CoV-2 spike protein under non-thermal microwave radiation. Sci Rep. 2021; 11(1):23373.
13. E. T. Chen, J. T. Thornton, S-H. Duh And P. T. Kissinger, Observation of Fermi Arc Surface States Induced by Organic Memristive/Memcapacitive Devices with a Double-Helical Polarized Single-Wall Nanotube Membrane for Direct Chelating with Matrix Matelloproteinase-2, Sensors and Transducers Journal, 214(7), 69-84, 2017.
14. E. T. Chen and H. Pardue, Analytical applications of catalytic properties of modified cyclodextrin, 2563-2567, 1993.
15. E. T. Chen, J. T. Thornton, S-H. Duh, Increase ACE2 Concentrations Embedded in the Memristive Electrochemical Sensor Membranes Promote Direct Reagent-free Sensing of S1 Antigen of SARS-Cov2 Virus, TechConnect Briefs in Biomaterials, Diagnostic and Bioimages, 111-114, 2022.
16. M. Ternes, M. Pivetta, F. Patthey, and W-D Schneider, Creation, electronic properties, disorder, and melting of two-dimensional surface-state-mediated adatom, Progress in Surface Science 85, 1-27, 2010.
17. C. Guang, R. D. Phillips, B. Jiang, Three key proteases-angiotensin-I-converting enzyme (ACE), ACE2 and renin—within and beyond the renin-angiotensin system, Arch Cardiovasc Dis. 2012, 105(6): 373-385.
18. P. Towler, B. Staker, S. G Prasad, ACE2 X-ray structures reveal a large hinge-bending motion important for inhibitor binding and catalysis, J. Biol Chen 2004, 23,279(17):17996-8007
19. F. Loder, A. P. Kampf, T. Kopp, et al., Magnetic Flux Periodicity of h/e in Superconducting Loops, Nature Physics, 2008, 4, 112-115.
20. E. T. Chen, J. T. Thornton, P. T. Kissinger, and S-H. Duh, Utilization of the Flexible Fractional Josephson Toroidal Arrays for Sensing, Memory Storage and Quantum Computing, Sensors and Transducers, 2018, 228(12), 30-47, 2018.
21. E. Wolf, G. Arnold, M. Gurvitch, and J. Zasadzinski, Josephson Junctions, History, Devices, and Applications, Pan Stanford Publishing Pte, Ltd. 2017.
22. M. Wang, R. Cao, L. Zhang, et al. Remdesivir and chloroquine effectively inhibit the recently emerged novel coronavirus (2019-nCoV) in vitro. Cell Res. 2020; 30(3): 269-271.
23. M. Di Ventra and Y. V. Pershin On the physical properties of memristive, memcapacitive, and meminductive systems, Nanotechnology 24, 255201, 2013.
24. M. Di Ventra, Y. V. Pershin, Memristors and Memelements: Mathematics, Physics and Fiction, Springer Nature, ISBN: 978-3-031-25624-0, 2023
25. P. Pfeiffer I. K. Egusquiza, M Di Ventra, M. Sanz, E. Solano, Quantum memristors, Scientific reports 6 (1), 29507, 2016.
26. E. T. Chen, J. T. Thornton, P. T. Kissinger and S-H. Duh, Utilization of the Flexible Fractional Josephson Toroidal Arrays for Sensing, Memory Storage and Quantum Computing, Sensors and Transducers Vol. 228, Issue 12, 30-47, 2018.
27. E. T. Chen, Chapter 13, Room-Temperature Self-Powered Nanostructure Organo-Metallic Superconductive Devices in the book of Nanoengineering, Quantum Science, and, Nanotechnology Handbook edited by Sergey Edward Lyshevski, CRC Press, ISBN Number: 978-0-367-19751-3 (Hardback). Published on Dec. 2, 2019.
28. E. T. Chen, J. T. Thornton, S-H. Duh published an article in the title of Delocalized Electron Relay in Superconductive Multiple-layer Self-Assembled Membranes Promoted Room-temperature d Wave Mixed-spin Triplet of Cooper-pair to Penetrate Toroidal Josephson Junction Barriers with a Magnitude Higher Open Circuit Potential, TechConnect Briefs in Sensors, MEMs, Electronics, 187-190, 2022.
29. E. T. Chen, Scale-up toroidal array quantum processing memory device with controllable and adjustable state-switch valves of making and applications thereto, Issued U.S. Pat. No. 11,351,924 B2 on 2022-12-20.
30. E. T. Chen, Nanostructured organic memristor/memcapacitor of making with an embedded low-to-high frequency switch and a method of inducing an electromagnetic field, U.S. Pat. No. 9,793,503 B2 on 2017-10-17.
31. B. Weiss, G. Ganepola, H. Freeman, Y. Hsu and M. Faupel, Breast Disease, 1994, 7(2), pp 91.
32 S. V. Sree, E. Ng, G. Kaw, R. Acharya-U, and B. Chong, J. of Medical Systems, 2011, 35(1). 79.
33. A. M. Hassan and M. Ei-Shenawee, Review of electromagnetic techniques for breast cancer detection, IEEE Transactions in magnetic, on line publisher, 2011.
34. E. T. Chen1, Y. Shen J. Thorten, C. Ngatchoul, S-H. Duh, and P. T. Kissinger, A Nanopore Biomimetic device quantitatively detects early stage cancer cells; a Contour Map Multiple Variable Correlation Method assesses the heat of cancer cells released, Techconnect Briefs, Nanotech 2012, pp 198-201.
35. E. T Chen, J. Thorten, C. Ngatchou, S-H Duh and P. Kissinger, Nanostructured Biomimetic Memristor Sensors Used for Therapeutic Monitoring of The Brain Cancer, NSTi-Nanotech, 2014, 2, 200-203.
36. E. T. Chen, Book chapter of Nanobiomimetic sensors and technology, Dekker Encyclopedia of Nanoscience and Nanotechnology Mar. 8, 2013.
37. L. Azzi, V. Maurino, A. Baj et al., Journal of Dental Research, 2020, 1-9. DOI:10.1177/0022034520969670
38. J. Song, Y. Li, X. Huang, et al., Systematic analysis of ACE2 and TMPRSS2 expression in salivary glands reveals underlying transmission mechanism caused by SARS-CoV-2, J Med Vial 92, 2556-2566, 2020.
39. E. T. Chen, Nanostructured biomimetic protein superconductive devices of making and its multiple applications thereto, U.S. Pat. No. 11,708,595, B2, Jul. 25, 2023.
40. E. T. Chen, J. T. Thornton and S-H. Duh, 3D-Cage Structured Biomimetic MMP-2/HSP60-Like Organo-Metallic Devices Sensitively Response to ATP Concentration Change Used for Defining the Anti-inflammatory and Pro-inflammatory Status, Sensors & Transducers, 2019, 236 (8), 28-39.

What is claimed is:

1. A nanostructured biomimetic angiotensin-converting enzyme 2 (ACE2) sensor comprising:
(a) an electrode has an organometallic superconductive membrane by self-assembling (SA) having arrays of Josephson toroidal junctions (JTJ);
(b) wherein the superconductive membrane has comprised a direct electron-relay comprising of a biomimetic ACE2 membrane SAM on a first layer of a conductive organic SAM comprising of nano-island nanostructured, and an analyte formed chelating coordinating bounds, forming a long-range direct electron-relay (DER) chain; and
(c) wherein the organometallic superconductive Josephson toroidal array membrane ACE2 sensor becomes a superconductive anharmonic oscillator at zero-bias potential.

2. The nanostructured biomimetic ACE2 sensor according to claim 1, wherein the superconducting SAM has a superconductive-insulator-superconductive (SIS) configuration as (SIS) with zinc atoms serve as a junction barrier in the JTJ array (JTJA).

3. The nanostructured biomimetic ACE2 sensor according to claim 1, wherein the superconductive JTJA membrane has Friedel-oscillation in the superlattice membrane.

4. The nanostructured biomimetic ACE2 sensor according to claim 1, wherein function groups of the superconductive TJJA membrane mimic a function of a zinc-finger of a native ACE2 protein, which is a receptor-binding domain (RBD) to attract a spike protein of a SARS-CoV-2 virus.

5. The nanostructured biomimetic ACE2 sensor according to claim 1, wherein Sensor 1 is orders of magnitudes sensitive to quantitative detect the S1 SARS-CoV2 virus over 40 aM to 120 nM concentrations without an inhibitor compared with the sensitivity of a native ACE2 electrochemical sensor under an antibody-free and labeling-free conditions using a Cyclic Voltammetry (CV) method.

6. The nanostructured biomimetic ACE2 sensor according to claim 1, wherein the sensor under the impact of 0.8 nM Remdesivir (REM) inhibitor 100% blocked S 1's biocommunication over the same S1 concentration range related to the sensitivity from the biomimetic sensor 1 control; and compared to the native ACE2 sensor 2, which 0.8 nM REM reduced the S1 sensitivity by 90.9% related to Sensor 2 without an inhibitor using the CV method.

7. The nanostructured biomimetic ACE2 sensor according to claim 1, wherein the sensor under the impact of 0.8 nM ABS02 inhibitor, it blocked S1's biocommunication 100% over the same S1 concentration range related to the sensitivity of the biomimetic sensor 1 control; and compared to the native ACE2 sensor 2, which 0.8 nM ABS02 reduced the S1 sensitivity by 100% related to Sensor 2 without an inhibitor using the CV method.

8. The nanostructured biomimetic ACE2 sensor according to claim 1, wherein the S1 virus inhibitor ABS02 is a 3D cage-structured inhibitor comprising supramolecules of bis imidazole modified β-dimethyl cyclodextrin (bM-β-DMCD, molecular formular: C₆₆H₁₁₀O₃₅N₄·18H₂O with a MW=1843.6), and a mono imidazole modified β-dimethyl cyclodextrin (mM-β-DMCD, the molecular formular: C₆₁H₁₀₄O 35N₄·4H₂O with a MW=1497), triacetyl-β-cyclodextrin (TCD), polyethylene glycol diglycidyl ether (PEG), poly (4-vinyl pyridine) (PVP), cysteine, zinc chloride, and collagen-1 cross-linked.

9. The nanostructured biomimetic ACE2 sensor according to claim 1, wherein Sensor 1 for without an inhibitor, has 96.4% reduced the sensitivity as far as the concerns of S1 virus gaining eternal equilibrium energy, compared with Sensor 2, the native ACE2 sensor 2, over 40 aM-120 nM (n=18), and 4 aM-120 nM (n=21), respectively with each sample monitored 120 s using the OPO method.

10. The nanostructured biomimetic ACE2 sensor according to claim 1, wherein under the impact of 0.8 nM REM inhibitor, the S1 virus intrinsic energy gaining rate increased by 63% detected by Sensor 1 over 400 aM-120 nM (n=15), but reduced by 99.17% detected by Sensor 2 (n=18) over 40 aM-120 nM with each sample monitored 120 s by the OPO method.

11. The nanostructured biomimetic ACE2 sensor according to claim 1, wherein under the impact of 0.8 nM ABS02 inhibitor, the S1 virus intrinsic energy gaining rate reduced by 100% detected by Sensor 1 (n=15) over 400 aM-120 nM, and it reduced the S1 virus energy gaining rate by 100% detected by Sensor 2 (n=12) over 40 aM-120 nM by the OPO voltage method.

12. The nanostructured biomimetic ACE2 sensor according to claim 1, wherein the device is a mems-element-superconductive quantum interference device (MEML-SQUID) with a switchable state valve between mems-element state and quantum superconductive state, that when a suitable S1 virus inhibitor as the valve appears in the presence of 3D-cage structured JJA membrane caused a high Josephson frequency oscillation, and it turns an i-V curve at a MEMS-state to an i-V curve of supercurrent at zero-bias state at high frequency at room temperature.

13. The nanostructured biomimetic ACE2 sensor according to claim 1, wherein the device further comprising of multiple-functioning of monitoring the normality of a cell reversible membrane potential (RMP) for the S1 SARS-CoV2 virus concentration effect between 40 aM to 120 nM using a Double-step Chronopotentiometry (DSCPO) or voltage method at J10 nA with each potential step at 0.25 Hz.

14. The nanostructured biomimetic ACE2 sensor according to claim 1, wherein the device further comprising of multiple-functioning of monitoring clinical normality of a ratio of a cell action potential vs. resting potential (Ap/Rp) in the S1 SARS-CoV2 virus concentration over 40 aM to 120 nM with 100% results fall in the safety zone in the presence of 0.8 nM ABS02 (n=15) and 0.8 nM REM (n=15), respectively, but 50% results for S1 virus (n=18) fall outside of the safety zone for the case of without an inhibitor.

15. The nanostructured biomimetic ACE2 sensor according to claim 1, wherein the device's results are traceable to use human fasting saliva samples with or without spiked 40 aM S1 virus showed a recovery rate (accuracy) is 96±4% using In OPO method. The recovery results showed 99-2% using the NIST SRM965 human serum with a certified glucose 300 mg/dL as the control, compared with spiked S1 antigen 60 nM in the serum by the OPO method.

16. The nanostructured biomimetic ACE2 sensor according to claim 1, wherein the device's results are traceable to the fasting salivary samples showed the recovery rate is 92.3±9% by spiked S1 antigen 40 aM samples against the saliva controls after corrected the factor between the saliva controls and the buffer controls using a voltage method. The recovery results showed 118±0.2% using the NIST SRM965 human serum with a certified glucose 300 mg/dL as the control, compared with spiked S1 antigen 60 nM in the serum by the DSCPO method.

17. The nanostructured biomimetic ACE2 sensor according to claim 1, wherein the device utilizes the toxic S1 SARS-COV2 virus protein as the secondary Josephson toroidal junction insulator, that promotes Cooper-pair long-range tunneling that induced a single flux quantum with the S-I-S·S1 virus·S1 inhibitor configuration in the presence of a S1 virus inhibitor at zero-bias, which the Josephson coupling energy induced supercurrent high frequency oscillation effectively deactivate the S1 SARS-COV2 virus without using an external magnetic power to deactivate the virus compare to the prior art. The invention provided a tool of fast screening a suitable virus inhibitor was demonstrated.
